(19) United States
(12) Patent Application Publication
(10) Pub. No.: US 2017/0102380 A1
(43) Pub. Date: Apr. 13, 2017

(54) METHOD FOR PHOTO-IMMOBILIZING BIOMOLECULES ON A NON-FUNCTIONALIZED CARRIER

(71) Applicant: COMMISSARIAT A L'ENERGIE ATOMIQUE ET AUX ENERGIES ALTERNATIVES (CEA), Paris (FR)

(72) Inventors: Julie CREDOU, Cachan (FR); Thomas BERTHELOT, Les Ulis (FR); Rita FADDOUL, Aix-en-Provence (FR)

(21) Appl. No.: 15/122,470

(22) PCT Filed: Mar. 5, 2015
(86) PCT No.: PCT/EP2015/054655

§ 371 Date: Aug. 30, 2016

(30) Foreign Application Priority Data
Mar. 5, 2014 (EP) 14157944.1
Jul. 31, 2014 (EP) 14179401.6
Aug. 8, 2014 (EP) 14180423.7

(51) Int. Cl. G01N 33/548; G01N 33/545; C07K 17/12

(52) U.S. Cl. CPC G01N 33/548; C07K 17/12; G01N 33/545

(57) ABSTRACT

The immobilization of biomolecules on a non-functionalized carrier by irradiating the biomolecule-impregnated carrier with a light of a wavelength of at least 340 nm.

### FIELD OF INVENTION

The present invention relates to a method for immobilizing biomolecules on non-functionalized carriers by irradiating the biomolecule-impregnated carrier with a light of a wavelength of at least 340 nm.

### BACKGROUND OF INVENTION

In various domains such as clinical diagnosis, drug screening, food quality control, environmental monitoring, there is a need to easily and rapidly detect biomolecules that are free in solution or attached to the surface of a cell, a virus or a bacteria. The biomolecules to be detected are essentially composed of proteins or peptides, such as enzymes, antibodies, receptors, transcription factors, hormones, and the like. In the field of clinical diagnosis, the identification of proteins in blood, urine or biopsy samples can for instance be useful for detecting bacterial or viral infections, cancers, diabetes, hormonal imbalance or pregnancy. Detecting pathogenic microorganisms may also be of primary importance for environmental purposes, and may be implemented for controlling the quality of the water and/or food.

Several methods have been developed for manufacturing biosensors, biochips, microarray and other immunoassay devices. It may, for instance, be noticed that blood glucose sensors, pregnancy tests, or urine test strips are among the most commonly distributed devices for identifying biomolecules.

The preparation of efficient bioassay devices, and more specifically of immunoassay devices, requires the robust immobilization of a large number of biomolecules of interest on a carrier. As a result of its improved capacity to immobilize proteins, and more specifically antibodies, by electrostatic interaction with the nitro functions displayed on its surface, nitrocellulose constitutes the most commonly used carrier material for preparing immunochromatographic devices. Nevertheless, nitrocellulose is an expensive, fragile and inflammable material, which was furthermore shown to be incompatible with newly developed multiplex biosensors such as lab-on-paper devices, microfluidic paper analytical devices (μPADs), or other paper-based analytical devices. Moreover, some agents such as spores and some bacteria may have difficulty in migrating along nitrocellulose. For these reasons, alternative carriers to nitrocellulose are sought by several teams.

Several methods for immobilizing biomolecules on carriers are known in the art, which may be classified in three major families: (i) physical methods, wherein the biomolecule is retained on the carrier through physical forces such as electrostatic, Van der Waals, hydrophobic interactions and the like; (ii) biological or biochemical methods wherein the biomolecule is linked to the carrier through biochemical affinity between two components (e.g. metal/ligand, ligand/protein, protein/antibody, etc.); and (iii) chemical methods, wherein covalent bonds maintain the biomolecule on the carrier. Nevertheless, each of these methods also display specific drawbacks.

Physical methods may be implemented through simple, rapid and cost-saving procedures, and advantageously limit the necessity for modifying the biomolecule or its carrier. Nevertheless, the weak and non-permanent interaction maintaining the biomolecule on the carrier also represent a major drawback of these methods, since biomolecules are progressively torn out, thus triggering a loss of activity of the corresponding biosensor.

Biological methods advantageously allow the biomolecules to be immobilized in a specific orientation, through reversible non-covalent, whereas strong and specific, interactions with the carrier. Nevertheless, these methods require complex and expensive procedures wherein the biomolecule and/or the carrier are modified for introducing a binding conjugate or a binding domain therein.

Finally, chemical methods provide a strong, stable and permanent coupling of the biomolecule to its carrier. The thus-conceived biosensors are robust and provide reproducible results. Nevertheless, the chemical treatments performed may modify and alter the structure and/or the activity of the biomolecules. The resulting biosensors, while reusable, may thus lack sensitivity as a consequence of biomolecule alteration.

Among the known covalent coupling techniques, photo-immobilization is probably the simpler and the faster to be implemented in a process for preparing bioassay devices and more specifically immunoassay devices: the carrier is generally coated with a photoreactive compound and the biomolecule of interest is covalently affixed to the carrier through photoactivation. Considering that short-wave UV (ultraviolet) light (i.e. 100 nm-400 nm) is known to alter biomolecules, photoimmobilization is then regularly performed under long-wave UV light (340 nm-400 nm) or visible light (400 nm-800 nm) (Viel et al., 2013, Langmuir 29:2075-2082; Volland et al., 2004, 34: 737-752). Nevertheless, all the photoimmobilization methods described so far require to use a photoreactive coupling intermediate, and further require the functionalization of the carrier through harsh conditions, in organic solvents, or with highly toxic reagents or side products.

There is therefore an ongoing need for cost-saving and rapid methods allowing bioassay devices, and more particularly immunoassay devices, to be prepared by immobilizing biomolecules on carriers through robust and sustainable binding. There is indeed a long-felt need for immobilization methods displaying a limited number of steps, allowing to save significant amounts of reactants solvents or adjuvants, and at the same time capable to preserve the activity of the biomolecules of interest through the use of mild-coupling reaction conditions.

Further, though paper-based immunoassay such as dipstick tests or lateral flow immunoassays (LFIAs) have been marketed and extensively employed for point-of-care (POC) diagnostics and pathogen detection since the 80s (diabetes and pregnancy tests being the most famous), the recent impetus given to paper-based microfluidics by American, Canadian and Finnish research teams has resulted in the development of new paper-based bioanalytical devices with complex designs allowing multiplex diagnosis.

Regarding the device shaping, the frame material of a multiplex device needs to be patterned with microfluidic channels. Thus, several methods for patterning carrier sheets, and more specifically paper sheets have been developed. Among the many processes are photolithography, using SU-8 or SC photoresist, “wax printing” or “wax dipping”, inkjet printing and laser cutting. With regard to the biosensing material, the spatially controlled immobilization of biomolecules is a key step in the development of biosensing devices.

Printing techniques such as micro-contact printing or inkjet printing are often preferred to spatially control biomolecule immobilization, since they allow quick cycles where only one step—printing biomolecule—is required. Moreover, printing is considered a biocompatible environmentally friendly process. It is a versatile technique enabling the deposition of variable kinds of solutions (biomolecules, polymers, solvents, metals) onto different types of substrates (cellulose, polymer, glass, silicon) and according to any design desired. It is a fast dispensing process allowing low-cost, high throughput fabrication, and therefore a very attractive approach regarding the economic and ecological goals. However, to be able to detect an immune answer, many printing cycles are needed so far. For example, 60 print cycles of an immune-sensing ink were necessary to detect 10 μg L⁻¹ (i.e. 10 ng mL⁻¹) of IgG molecule (Abe et al., Anal. Bioanal. Chem., 2010, 398, 885-893) and 24 cycles of protein ink were inkjet printed in order to detect 0.8 μM of human serum albumin (HSA) (i.e. 53.6 μg mL⁻¹ since M_{HSA}=67 kDa) (Abe et al., Anal. Chem. 2008, 80, 6928-6934). Moreover, printing is only a dispensing technique and is not sufficient by itself to strongly immobilize biomolecules onto carriers, and more specifically onto cellulose. Recent findings revealed that about 40% of antibody molecules adsorbed onto cellulose paper can actually desorb from the fibers (Jarujamrus et al., Analyst 2012, 137, 2205-2210). Direct adsorption of antibodies onto carriers, and more specifically, cellulose is therefore too weak to allow the permanent immobilization required in the development on immunoassay and carrier activation or functionalization is thus necessary.

The present inventors have surprisingly discovered a method allowing biomolecules such as proteins (e.g. antibodies) to be strongly immobilized onto a wide range of non-functionalized carriers, including cellulose, in absence of any photocoupling intermediate, upon exposure to a light of a wavelength of at least 340 nm. The process of the invention surprisingly and advantageously allows biomolecules to be immobilized on its carrier with a coupling efficiency similar to that observed for nitrocellulose, and unexpectedly preserves the said biomolecules from the loss of activity resulting from known chemically-mediated photoimmobilization methods. The process of the invention thus appears to be faster, cost-saving and environmentally-friendly, in particular when it is combined with inkjet printing techniques.

### SUMMARY

This invention thus relates to a process for immobilizing biomolecules on a non-functionalized carrier comprising the steps of:
(i) impregnating the non-functionalized carrier with a solution containing the said biomolecules; and
(ii) irradiating the impregnated carrier resulting from step (i) with a light of a wavelength of at least 340 nm;
   wherein said biomolecules are not functionalized.

In a particular embodiment, said non-functionalized carrier is selected from the group consisting in cellulose, polyethylene terephthalate (PET), polyethylene naphthalate (PEN), poly(methyl methacrylate) (PMMA), polyurethane (PU), poly(vinyl chloride) (PVC), polyethylene (PE), polystyrene (PS), polylactate, polyamide, and combinations thereof.

In a particular embodiment, the process of the invention further comprises a step of drying said impregnated carrier after step (i) and before step (ii).

In a particular embodiment, the process of the invention further comprises at least one step of washing the irradiated carrier resulting from step (ii).

In a particular embodiment, the light used for irradiating the impregnated carrier has a wavelength of from 340 nm to 800 nm, preferably of from 340 nm to 400 nm, preferably of from 400 nm to 800 nm, and has preferably a wavelength of about 365 nm.

In a particular embodiment, said impregnated carrier is irradiated during step (ii) with a photoenergy of from 1 mJ/cm² to 500 J/cm², preferably of from 1 J/cm² to 80 J/cm², and preferably of about 10 J/cm².

In a particular embodiment, said non-functionalized carrier is substantially non-porous.

In a particular embodiment, the process of the invention further comprises a preliminary step, before step (i), of rendering the said non-functionalized carrier substantially non-porous, comprising:
a) impregnating said non-functionalized carrier with at least one filler until saturation of the carrier, and
b) drying the impregnated non-functionalized carrier resulting from step a).

In a particular embodiment of the process of the invention, said non-functionalized carrier is cellulose and said at least one filler is selected in the group consisting of glucose, paraffin, sulfonated polymers, polyacrylic acid (PAA), poly-2-hydroxyethyl methacrylate (PHEMA), polymethyl methacrylate (PMMA), poly(ethylene glycol) dimethacrylate (PPEGDMA), polypropylene (PP), polys(styrene sulfonic acid-maleic anhydride), poly(vinyl phosphonic acid), polyethyleneglycol, salts thereof and/or combinations thereof.

In a particular embodiment, said non-functionalized carrier is in a form selected in the group consisting of a bead, a well, a sheet, a powder, a stick, a plate, a strip or a tube, and is preferably in the form of a sheet.

In a particular embodiment, said biomolecule is selected from the group consisting of proteins or peptides, such as antibodies, antigens, enzymes, transcription factors, protein domains or binding proteins.

In a particular embodiment, said biomolecule is displayed on the surface of a bacteria, a virus or a micro-organism, or is free in solution.

In a particular embodiment, the buffer used for washing the irradiated carrier is selected from the group consisting of water, phosphate buffer, carbonate buffer, borate buffer, HEPES buffer, MES buffer or any other aqueous biological buffer, and further optionally comprises salts and/or detergent.

The present invention further concerns a grafted carrier obtained by the process of the invention, wherein said grafted carrier comprises biomolecules immobilized thereonto.

The present invention further concerns a bioassay device comprising at least one grafted carrier according to the invention, wherein said bioassay device is preferably an immunoassay device such as an immunochromatographic strip or an immunochromatographic multiplex system.

The present invention further concerns the use of at least one grafted carrier according to the invention or of a bioassay device according to the invention for diagnosis, affinity chromatography, proteomics, genomics and/or drug screening.

The present invention also concerns a bioassay kit comprising at least one carrier as defined above or at least one bioassay device according to the invention.

### DETAILED DESCRIPTION

This invention therefore relates to a surprising and advantageous process allowing biomolecules to be immobilized on a non-functionalized carrier and comprising the steps of (i) impregnating the non-functionalized carrier with a solution containing the said biomolecules; and (ii) irradiating the impregnated carrier resulting from step (i) with a light of a wavelength of at least 340 nm; wherein said biomolecules are not functionalized.

Within the present invention, by “non-functionalized carrier” or “non-functionalized biomolecule”, it is meant that the said carrier or the said biomolecule are not modified to be rendered more photoactive. The said carrier or biomolecule are thus not modified for incorporating photoreactive moieties such as aryl azide moieties, halogenated aryl azide moieties, benzophenones moieties, moieties including diazo compounds, moieties comprising diazine derivatives, or nitrobenzyl moieties.

In a particular embodiment, the non-functionalized carriers for use in the method of the invention are selected from the group consisting in cellulose, polyethylene terephthalate (PET), polyethylene naphthalate (PEN), poly(methyl methacrylate) (PMMA), polyurethane (PU), poly(vinyl chloride) (PVC), polyethylene (PE), polystyrene (PS), polylactate, polyamide, and combinations thereof.

Within the present invention, by “cellulose”, it is meant a polysaccharide of formula (a) (see below), consisting of a linear chain of several hundred to over ten thousand β(1-4) linked D-glucose units.

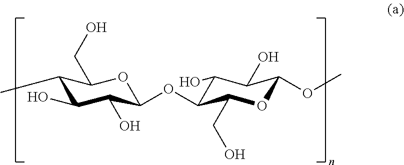

Cellulose for use within the present invention may be obtained from any origin, such as green plants, algae, oomycetes or bacteria. Cellulose for use in the present invention is non-functionalized, i.e. it has not been treated chemically for generating reactive moieties. In a preferred embodiment, cellulose for use in the present invention is pure cellulose paper. Depending on the destination of the grafted cellulose carrier prepared according to the process of the present invention, the density and/or shape of the cellulose carrier may vary. Cellulose is an affordable biopolymer, which is also biocompatible, biodegradable and easily available. Cellulose is of specific interest since it exhibits wicking properties allowing biomolecules in solution to migrate by capillarity without needing any external power sources. It is further available in a broad range of thickness and possesses well-defined pore sizes, is easy to store and safely disposable.

In a particular embodiment, the non-functionalized carriers for use in the method of the invention are porous carriers, such as cellulose. In another particular embodiment, the non-functionalized carriers for use in the method of the invention are substantially non-porous carriers.

Within the present invention, by “substantially non-porous carrier”, it is meant that the carrier is not permeable to water or other fluids. In a particular embodiment, the carrier used in the method of the invention is a naturally substantially non-porous carrier. Naturally substantially non-porous carriers for use in the present invention are preferably selected in the group consisting of polyethylene terephthalate (PET), polyethylene naphthalate (PEN), poly(methyl methacrylate) (PMMA), polyurethane (PU), poly(vinyl chloride) (PVC), polyethylene (PE), polystyrene (PS), polylactate, polyamide (PA), and combinations thereof.

In another embodiment of the invention, the carrier for use in the method of the invention is a naturally porous carrier that has been pretreated for rendering it substantially non-porous. In a particular embodiment, suitable porous carriers for use in the present invention comprise cellulose or derivatives of cellulose. Other suitable porous carriers comprise aerogel, porous polymers or porous membranes such as for instance PVDF membranes, PE membranes, Teflon membranes and polycarbonate membranes, on non water-soluble polysaccharides such as lignin, pectin, starch, dextran or chitosan.

In a particular embodiment, the said naturally porous carriers are rendered substantially non-porous by impregnating said carrier with at least one filling substance.

The present invention thus also concerns a process of preparing a substantially non-porous carrier starting from a porous material, and comprising the steps of:
a) impregnating said porous non-functionalized carrier with at least one filler until saturation of the carrier; and
b) drying the impregnated carrier resulting from a).

In a particular embodiment of the invention, step a) can be performed by any technique known in the art.

In a particular embodiment, the porous carrier is immerged in at least one filling substance solubilized in a suitable solvent. In a particular embodiment, the carrier is immerged for a period ranging from 1 hour to 24 hours. In another embodiment, the filling substance solubilized in a suitable solvent is allowed to flow through the carrier, e.g. under vacuum aspiration, until not outflow (or an extremely low outflow) is observed. In a particular embodiment, the carrier is impregnated with a solution comprising at least one filling substance or with a combination of filling substances. In a particular embodiment, the carrier is successively impregnated with several filling solutions comprising at least one filling substance or a combination of filling substances. In a particular embodiment, the carrier may be successively impregnated with filling substances solubilized in aqueous solvents and/or in organic solvents, provided that the organic solvents used for solubilizing the filling substance are water-miscible. In a particular embodiment, when filling substances are solubilized in water-immiscible organic solvents, the carrier is first impregnated with filling substances solubilized in aqueous solvents, then fully dried, before being impregnated with filling substances solubilized in water-immiscible organic solvents.

Within the present invention, by “filling substance” or “filler”, it is meant a substance capable of substantially filling the pores of a porous carrier as defined above. Suitable fillers for use in the present invention comprise those that can be solubilized in an aqueous solvent, those that can be solubilized in a water-miscible organic solvent and those that can be solubilized in water-immiscible organic solvents. In a particular embodiment, fillers for use in the method of the invention are selected in the group consisting of glucose; paraffin; sulfonated polymers such as for example polystrene sulfonic acid and its derivatives, sulfonated polyacrylic acid and its derivatives; polyacrylic acid (PAA); poly-2-hydroxyethyl methacrylate (PHEMA); polymethym methacrylate (PMMA); poly(poly(ethylene glycol) dimethacrylate) (PPEGDMA); polypropylene (PP); poly(styrene sulfonic acid—Maleic anhydride); poly(vinyl phosphonic acid); polyethyleneglycol; as well as salts thereof and/or combinations thereof. In a preferred embodiment, glucose is used as a filler.

In a particular embodiment, when water-soluble substances are used for filling porous carriers such as cellulose, the concentration of such fillers ranges from 1 mg/mL to 1000 mg/mL, preferably about 100 mg/mL. In a particular embodiment, porous carriers saturated with water-soluble substances are impregnated for a time comprised between 0 min and 24 hours, or comprised between 0 min and 1 night. Water-soluble substance for use in the present invention is advantageously glucose or any other saccharide (such as, for example, fructose, lactose and the like). In one embodiment, the water-soluble substance is glucose.

In another particular embodiment, when water-miscible substances are used for filling porous carriers such as cellulose, the concentration of such fillers ranges between 0.1 mg/mL and 1000 mg/mL. In a particular embodiment, porous carriers saturated with water-miscible substances are impregnated for a time comprised between 0 min and 24 hours, or comprised between 0 min and 1 night. Water-miscible substance for use in the present invention is advantageously polyacrylic acid, poly(styrenesulfonic acid-Maleic anhydride), or poly(ethyleneglycol).

In another particular embodiment, when water-immiscible substances are used for filling porous carriers such as cellulose, the concentration of such fillers ranges between 0.1 mg/mL and 100 mg/mL, preferably about 10 m/mL. In a particular embodiment, porous carriers saturated with water-immiscible substances are impregnated for a time comprised between 0 min and 24 hours, or comprised between 0 min and 1 night. Water-immiscible substance for use in the present invention is advantageously paraffin or polystyrene or wax (such as, for example, bee wax, carnauba wax and the like). In one embodiment, the water-immiscible substance is paraffin or polystyrene.

Depending on the destination of the grafted carrier prepared according to the process of the present invention, the density and/or shape of the carrier may vary. Shapes suitable for the grafted carrier of the invention include but are not limited to: a bead, a well, a sheet, a powder, a stick, a plate, a strip, a tube, as well as any 3D shape alternative thereof. In a particular embodiment of the invention, cellulose is provided under the form of a sheet, also known in the art under the term “paper”.

Within the present invention, by “impregnating”, it is meant that biomolecules, in suspension in a solvent, are dispensed onto the non-functionalized carrier by any method known in the art, such that the said biomolecules in solution are placed into contact with the surface of the carrier. The biomolecule solution may thus be dispensed onto the carrier under the form of drops, or droplets, such as for instance those formed by a printing system or by printing techniques such as silkscreen printing, inkjet printing, spraying, spin coating, and the like. In a preferred embodiment, the biomolecule solution is dispensed onto the carrier by inkjet printing. In another embodiment, the carrier is immerged in a solution comprising the said biomolecules.

Within the present invention, by “irradiating”, it is meant subjecting the non-functionalized carrier impregnated with the solution of biomolecules to a light having a wavelength of at least 340 nm. In a preferred embodiment, the light used for irradiating the impregnated carrier has a wavelength of from 340 nm to 800 nm (long-scale UV and visible light). In another embodiment, the light used for irradiating the impregnated carrier has a wavelength of from 400 nm to 800 nm (visible light). In a preferred embodiment, the light used has a wavelength of about 365 nm.

Within the process of the invention, irradiation is conducted for a period of time and with a wavelength that are suitable for subjecting the impregnated carrier to a photoenergy of from 1 mJ/cm² to 500 J/cm², preferably of from 1 J/cm² to 80 J/cm². In a preferred embodiment, irradiation is conducted for a period of time and with a wavelength that are suitable for subjecting the impregnated carrier to a photoenergy of about 10 J/cm².

Within the process of the invention, irradiation is conducted for a period of time of at least 1 second, preferably from 16 to 1280 minutes, preferably of about 160 minutes.

Within the present invention, by “immobilized”, it is meant that moieties of the biomolecules of interest present in the solution used for impregnating the non-functionalized carrier are covalently coupled to moieties of the carrier surface molecules, by co-chemical reaction triggered by irradiation. As a result of the process of the invention, the biomolecules of interest are thus covalently coupled to the carrier, while having preserved 80%, preferably 90%, preferably 99% and more preferably 100% of their biological activity.

Within the present invention, by “biomolecule”, it is meant any biological molecule selected from the group consisting of proteins or peptides, such as antibodies, antigens, enzymes, transcription factors, protein domains or binding proteins. In a preferred embodiment, the biomolecules for use in the present invention are antibodies and proteins. The biomolecules for use in the present invention are not modified either through chemical processes or by genetic engineering for improving their capacity to bind the carrier, by the introduction of photoreactive moieties.

In another preferred embodiment, the biomolecules for use in the present invention are modified, for example through the addition of a labelling system. In a preferred embodiment, the biomolecules of interest may correspond to biomolecules involved into biological membranes such as transmembrane proteins or antigenic proteins or peptides displayed on a biological membrane. In a particular embodiment of the present invention, the biomolecules of interest may be involved in the biological membrane of a cell, in the outer membrane or in the cell wall of a bacteria or a virus. In a preferred embodiment, by a solution of “biomolecules”, it is also meant a solution wherein the biomolecules of interest are provided on the membrane, cell wall or envelope of a cell, a bacteria or a virus, together with the said cell, bacteria or virus. In another embodiment, the biomolecules for use in the present invention correspond to biomolecules which are not excreted by cells such as non-excreted proteins. In another embodiment, the biomolecules for use in the present invention are free in solution, and may have been either purified from a biological organism or synthesized in vitro. For use in the method of the invention, the biomolecules are solubilized, or suspended, in a solvent including but not limited to: water, water for injection, phosphate buffer, carbonate buffer, borate buffer, HEPES buffer, MES buffer or any other aqueous biological buffer.

Within the present invention, by “drying”, it is meant evaporating the solvent wherein the biomolecules of interest are solubilized, thus allowing the corresponding biomolecules to be concentrated on the surface of the carrier. In a preferred embodiment, the carrier impregnated with the solution containing the biomolecules of interest is dried before the irradiation step. Drying may be performed by any mean known in the art, and in particular in subjecting the impregnated carrier to a source of heating, such as for instance, a ventilated oven.

In a particular embodiment of the present invention, a drying step is implemented before the irradiation step when said irradiation step (ii) is performed with a photoenergy of at least 1 mJ/cm², preferably of at least 1 J/cm², and preferably of about 10 J/cm².

Within the present invention, by “washing”, it is meant applying a liquid on the irradiated carrier (i.e. after the irradiation step) for removing any unbound biomolecule. According to the process of the invention, the washing step may be performed with any solvent which does not alter the structure or the biological activity of the immobilized biomolecules and which does not alter the carrier. Preferred washing solutions for the use in the present invention include but are not limited to: water, water for injection, phosphate buffer, carbonate buffer, borate buffer, HEPES buffer, MES buffer or any other aqueous biological buffer, optionally enriched with salts and/or detergent.

Another object of the present invention concerns a grafted carrier obtained by the process of the invention wherein said carrier comprises biomolecules immobilized thereonto.

Within the present invention, by “grafted carrier”, it is meant a carrier wherein biomolecules of interest were immobilized through the process of the invention, i.e. though irradiation at a wavelength of at least 340 nm. As a result of the process of the invention, the said grafted carrier is thus composed of biomolecules of interest which are covalently linked to a carrier absent any chemical intermediate or adjuvant, and absent any functionalization of the said biomolecules.

Another object of the present invention concerns a bioassay device, preferably an immunoassay device, such as for instance an immunochromatographic strip or an immunochromatographic multiplex system.

Within the present invention, by “bioassay device”, it is meant any device intended for detecting and/or quantifying biological or non-biological compounds, objects or organisms, including at least one grafted carrier of the invention.

Within the present invention, by “immunoassay device”, it is meant any device intended for immunodetection or immunoquantification purposes including at least one grafted carrier of the invention.

Within the present invention, by “immunochromatographic strip”, it is meant a device composed of a loading area, a detection area and an absorbent pad, the whole being affixed onto a plastic carrier. The detection area is formed by grafted carriers according to the invention. Migration is supported by two migration areas, surrounding the detection area, that are capable to trigger the sample to be tested by adsorption to the detection area.

Within the present invention, by “immunochromatographic multiplex system”, it is meant a system generally composed of hydrophilic paper channels, delimited by hydrophobic plastic stencils, such as those disclosed in patent applications filed under Nos. FR1161722 and FR1260083. The several levels of the system are stacked and connected together for forming a 3D fluidic system. A sample introduced at the top of the system is divided in as many aliquots as the number of channels, and is thus allowed to contact various biomolecules of interest immobilized on carriers according to the invention, and displayed in separated assay areas. An immunochromatographic multiplex system for instance allows different antigens to be detected within a same sample, using several carriers grafted with different antibodies.

Another object of the present invention concerns the use of at least one carrier according to the invention or of an immunoassay device according to the invention for diagnosis, affinity chromatography, proteomics, genomics and/or drug screening.

### BRIEF DESCRIPTION OF THE DRAWINGS

FIG. 1 is a photograph showing the localized immobilization of labelled antibodies through a mask. The localized immobilization is observed on the left on the functionalized cellulose (expected result), as well as on the right on pristine cellulose (unexpected).

FIG. 2 is a photograph showing the localized immobilization of murine antibodies. Murine antibodies immobilized on functionalized cellulose (on the right) or on pristine cellulose (on the left), are revealed by gold-labelled goat-anti-murine antibodies after saturation of the membrane with gelatin.

FIG. 3 is a photograph showing the influence of irradiation energy on antibody immobilization. OVA1 antibodies immobilized on nitrocellulose or cellulose, after optional drying (S) of the membrane, and irradiation (I) at 1 J/cm², 10 J/cm² or 80 J/cm², are revealed by gold-labelled OVA35 antibodies. In absence of OVA antigen (left panel), no signal is detected. In presence of OVA antigen (right panel), performances of nitrocellulose are reached for an irradiation energy of 10 J/cm². The results corresponding to 2 different samples are shown.

FIG. 4 is a histogram showing the specific colorimetric intensity obtained for lanes of the right panel of FIG. 3. FIG. 4 shows that the activity rate of antibodies adsorbed on nitrocellulose is reached on cellulose after drying and irradiating with 10 J/cm². The results corresponding to 2 different samples are shown, excepting for controls (nitrocellulose and cellulose).

FIG. 5 is a histogram showing the activity rate of antibodies immobilized on cellulose, after drying (S), irradiation (I) or drying and irradiation (S+I). The results of 2 different samples are presented for each condition.

FIG. 6 is a histogram showing the activity rate of antibodies immobilized on cellulose after drying (S), 2^{nd} column from the left, drying and irradiating at 365 nm (S+I@365), 3^{rd} column from left, and drying and irradiating with visible light (S+I@visible), 4^{th} column from left.

FIG. 7 is a histogram showing the activity rate of antibodies immobilized on cellulose without treatment (CF1, 1^{st} lane from left), after drying and irradiating (3^{rd} lane from left), or after drying and irradiating, then leaving in oven for 1 week at 40° C. (5^{th} lane from left). Results are compared to the activity rate measured after immobilization on nitrocellulose (2^{nd} lane from left), or on nitrocellulose after 1 week in oven at 40° C. (4^{th} lane from left).

FIG. 8 is a drawing showing the general structure of an immunochromatographic assay multiplex. Level 1 corresponds to the loading area. Level 2 shows the detection areas. The sample introduced on the top of the multiplex is divided into 4 aliquots directed towards the 4 assay areas of the multiplex. Papers grafted with different antibodies are introduced in the various assay areas. Level 3 shows the migration carriers. Levels 4 and 5 show the absorbent pads. Level 6 shows the supporting base.

FIG. 9 is a photograph showing the results of immunochromatographic assays conducted on multiplex systems. For each antigen tested, the coloration of the specific area where the corresponding specific antibody has been introduced is observed.

FIG. 10 is a schematic representation of the pattern printed with antibody solutions.

FIG. 11 is a schematic representation of the structure (a) and proportioning (b) of an immunochromatographic strip of an embodiment of the invention.

FIG. 12 is a photograph showing a photo-patterning obtained with gold-labeled goat anti-mouse tracer antibodies. Photographs were taken with a regular digital camera.

FIG. 13 is a photograph showing the influence of the dispensing process on biological activity and membrane VDL (Visual Detection Limit). The first set of strips (a) results from usual BioDot dispensing method, the second (b) from 1-layer inkjet printing, and the third (c) from 5-layer inkjet printing. Antibodies were adsorbed onto nitrocellulose and photoimmobilized onto cellulose. Their actual immobilization was confirmed thanks to gold-labeled goat anti-mouse tracer (control strips). The capture of OVA antigen by the immobilized antibodies was highlighted by gold-labeled murine anti-OVA tracer (OVA strips). The strips corresponding to the membranes VDL are labeled with a cross. Photographs were taken with the Molecular Imager. All experiments were reproduced 3 times but only one is shown here.

FIG. 14 is a scheme representing the detailed structure of an IgG antibody molecule (a) and general structure of an IgM antibody molecule (b).

FIG. 15 is a graph showing the antibody solutions viscosities at 24° C. and shear rate varying from 100 to 10 000 s⁻¹. The plain line corresponds to murine monoclonal antibody anti-OVA. Discontinued line corresponds to goat polyclonal antibody anti-mouse. The graph is expressed in viscosity (mPa s) as a function of Shear rate (s-1).

FIG. 16 is a scheme representing the molecular structure of the paper substrates (a) and filling substances (b) used in the examples.

FIG. 17 is a graph representing the XPS survey analysis of unprinted paper substrates. (a) is spectrum from nitrocellulose sheet, (b) from cellulose, (c) from glucose-cellulose and (d) from paraffin-cellulose. The peaks corresponding to O 1s, C 1s and N 1s orbitals are labeled.

FIG. 18 is a graph representing the IR spectra of unprinted paper substrates. (a) is spectrum from nitrocellulose sheet, (b) from cellulose, (c) from glucose-cellulose and (d) from paraffin-cellulose. All spectra have several bands in common which correspond to O—H, C—H, C—C, C—O and O—C—O stretching vibrations. The N—O stretching vibrations specific to nitrocellulose are labeled.

FIG. 19 is a graph representing the line profiles of the unprinted paper substrates.

FIG. 20 is a histogram representing the surface roughness (Ra) of the unprinted paper substrates.

FIG. 21 is a photograph representing SEM micrographs of unprinted nitrocellulose (a), cellulose (b), glucose-cellulose (c) and paraffin cellulose (d).

FIG. 22 is a graph representing the XPS survey analysis of antibody-printed paper substrates. (a) is spectrum from nitrocellulose sheet and (b) from cellulose. The peaks corresponding to O 1s, C 1s and N 1s orbitals are labeled.

FIG. 23 is a graph representing the IR spectra of antibody-printed paper substrates. (a) is spectrum from nitrocellulose sheet, (b) from cellulose, (c) from glucose-cellulose and (d) from paraffin-cellulose. All spectra have several bands in common which correspond to O—H, C—H, C—C, C—O and O—C—O stretching vibrations. The N—O stretching vibrations specific to nitrocellulose are labeled.

FIG. 24 is a photograph showing the influence of the substrate and its pretreatment on biological activity and membrane VDL. The first set of strips (a) is made of nitrocellulose, the second (b) of cellulose, the third (c) of glucose-cellulose and the fourth (d) of paraffin-cellulose. Antibodies were adsorbed onto nitrocellulose and photoimmobilized onto cellulose substrates. Their actual immobilization was confirmed thanks to gold-labeled goat anti-mouse tracer (control strips). The capture of OVA antigen by the immobilized antibodies was highlighted by gold-labeled murine anti-OVA tracer (OVA strips). The strips corresponding to the membranes' VDL are labeled with a cross. Photographs were taken with the Molecular Imager. All experiments were reproduced 3 times but only one is shown here.

FIG. 25 is a photograph showing the biological activity of antibodies printed according to a complex design. The first set of strips (a) was produced with nitrocellulose membrane and the second (b) with cellulose. Antibodies were adsorbed onto nitrocellulose and photoimmobilized onto cellulose. The capture of OVA antigen by the immobilized antibodies was highlighted by gold-labeled murine anti-OVA tracer. For each set of strips photographs were taken with both a digital camera (left pictures) and the Molecular Imager (right pictures).

FIG. 26 is a SEM photograph representing untreated CF1 cellulose sheet (see A)) and CF1 sheet treated with a solution of Poly(acrylic acid) sodium salt (250.000 (Sigma Aldrich)).

FIG. 27 is a SEM photograph representing a ×50 magnification (A) or a ×300 magnification (B) of a PET surface wherein a) corresponds to the virgin PET surface, b) corresponds to proteins printed on PET, c) corresponds to protein-printed PET surface washed with ultrasound for 10 s, and d) corresponds to protein-printed PET surface washed with ultrasound for 10 min.

FIG. 28 is a graph showing the IR result obtained with virgin PET, protein-printed PET, protein-printed PET washed with water and with ultrasound for 10 s or 10 min.

### EXAMPLES

The present invention is further illustrated by the following examples.

### Example 1: Localized Immobilization of Labelled Antibodies Through a Mask (Photolithography)

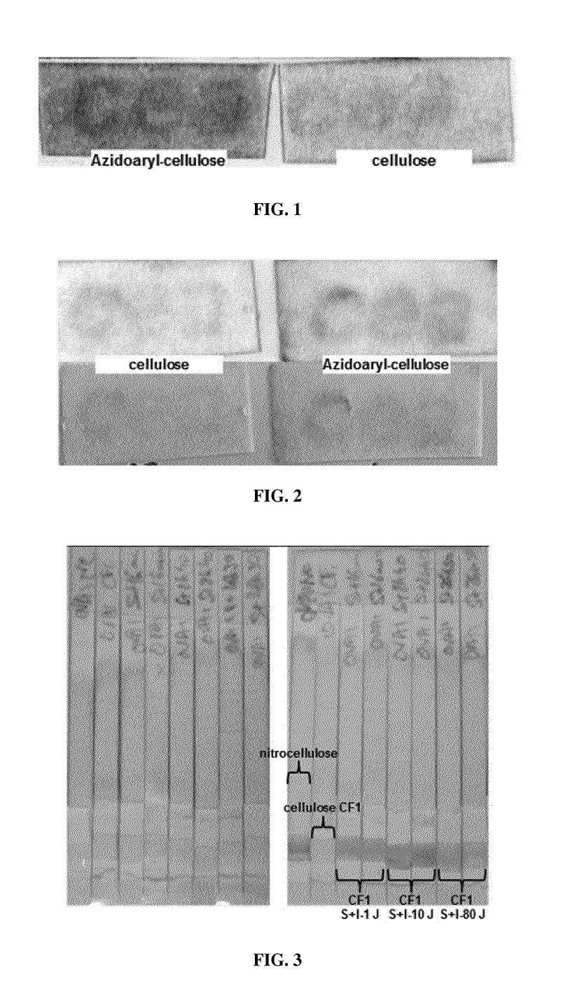

A solution of anti-murine goat antibodies, labelled with colloidal gold according to the standard method (Credou et al., 2013, J. Mater Chem. B, 1: 3277-3286) was diluted three times then poured on a 2 cm²-cellulose sheet (1×2 cm), at a rate of 20 μL/cm². Arylazide-functionalized cellulose was compared to pristine cellulose. Drying was deliberately omitted for avoiding important background noise resulting from the adsorption of gold particles. A mask was placed on the antibody-impregnated membrane and the system was irradiated for 80 minutes (at 5 J/cm²). After mask removal, samples were rinsed overnight with a phosphate buffer. Surprisingly, localized immobilization of antibodies was observed not only on the functionalized paper (expected result) but also on the pristine paper (unexpected result) (see FIG. 1).

### Example 2: Localized Immobilization of Murine Antibodies

Simple murine antibodies were immobilized for ensuring that the selective photoimmobilization observed in Example 1 did not result from colloidal gold particles interference. Murine antibodies, provided in solution at a concentration of 1 mg/mL in potassium phosphate buffer 0.1M, pH 7.4, were immobilized according to the process described in example 1. Membranes were saturated with gelatin, then the grafted antibodies were detected with anti-murine goat antibodies labelled with colloidal gold.

Antibody immobilization was observed on both functionalized and pristine papers (see FIG. 2).

### Example 3: Influence of Irradiation Energy

Anti-ovalbumin OVA1 antibodies were poured on CF1 cellulose sheets, and further concentrated by drying the impregnated paper (S). The system was then irradiated (I) at 365 nm for various times, corresponding to different energy levels: 16 min (about 1 J/cm²), 2 h40 (about 10 J/cm²) and 21 h20 (about 80 J/cm²). Finally, paper was rinsed 3 times for 5 minutes with phosphate buffer.

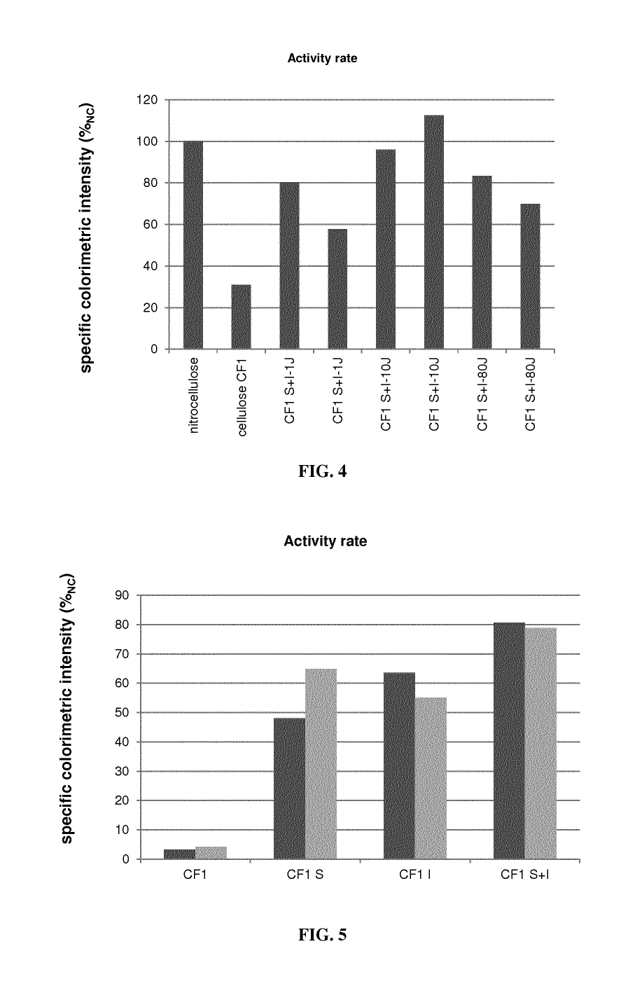

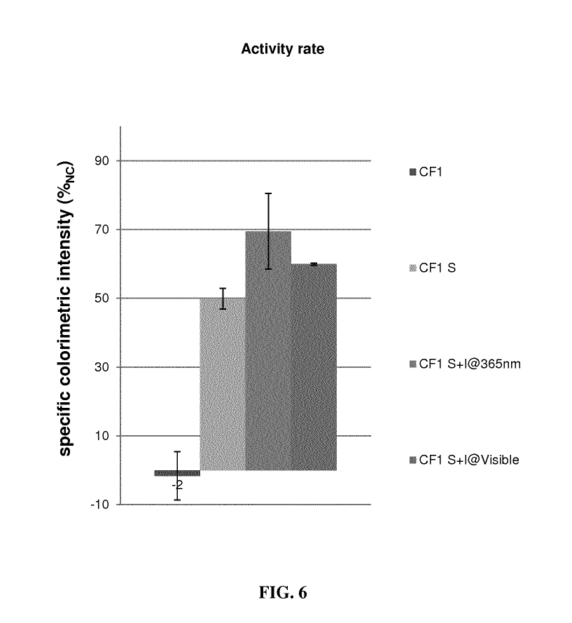

As can be seen in FIGS. 3 and 4, performances of nitrocellulose were reached with an irradiation energy of 10 J/cm², for both grafting rate and activity rate.

### Example 4: Influence of Drying Upon Short Irradiation Time

OVA1 antibodies were poured onto CF1 cellulose sheets. The impregnated papers were either dried for concentrating the antibodies (S) or left undried. Some dried and non-dried impregnated papers were then irradiated (I) at 365 nm for 16 min (about 1 J). Papers were rinsed with 3 successive baths (5 min each) in phosphate buffer. Grafting rate and activity rate were assessed.

Results indicate that drying appears to be required for short irradiation times (data not shown). Otherwise, antibodies remain in solution, too far away from fibers for ensuring a strong immobilization.

### Example 5: Influence of Drying Upon Long Irradiation Time

OVA1 antibodies were poured onto CF1 cellulose sheets. The impregnated papers were either dried for concentrating the antibodies (S) or left undried. Some dried and non-dried impregnated papers were then irradiated (I) at 365 nm for 2 h40 (about 10 J/cm²). Papers were rinsed overnight with phosphate buffer enriched in salts and detergent. Grafting rate and activity rate were assessed.

As can be seen in FIG. 5, drying appears to be beneficial, but might be omitted for long irradiation times. Indeed, for long irradiation times, drying occurs naturally in the course of irradiation thereby allowing antibodies to get closer to the fibers.

### Example 6: Influence of the Rinsing Solution

OVA1 antibodies were poured onto CF1 cellulose sheets. The impregnated papers were either dried for concentrating the antibodies (S) or left undried. Dried impregnated papers were then irradiated (I) at 365 nm for 2 h40 (about 10 J/cm²). Papers were rinsed either by 3 successive baths (5 min each) of phosphate buffer (P), or overnight with phosphate buffer enriched in salts and detergent (S/T). Grafting rate and activity rate were assessed.

Results indicate that extensive rinsing (e.g. overnight) with a phosphate buffer enriched in salts and detergent allows maintaining on the surface only molecules that are strongly immobilized (antibody adsorption is indeed reduced) (data not shown). The resulting signal appears to be slightly weaker, but results appear to be more reproducible.

### Example 7: Influence of Paper Nature

OVA1 antibodies were poured onto CF1, Chr1 or Xerox cellulose sheets. The impregnated papers were either dried for concentrating the antibodies (S) or left undried. Dried impregnated papers were then irradiated (I) at 365 nm for 2 h40 (about 10 J/cm²). Papers were rinsed extensively with phosphate buffer enriched in salts and detergent. Grafting rate and activity rate were assessed.

Results indicate that the process of the invention allows the observed signal to be increased independently from the nature of the paper, with respect to adsorption alone (data not shown). The Xerox paper is treated for being hydrophobic: the antibody solution is thus hindered to penetrate between the fibers, thereby explaining a lower grafting rate. The process of the invention thus allows a larger quantity of functional antibodies to be strongly immobilized on any type of cellulose.

### Example 8: Influence of the Wavelength

OVA1 antibodies were poured onto CF1 cellulose sheets. The impregnated papers were either dried for concentrating the antibodies (S) or left undried. Dried impregnated papers were then either irradiated (I@365) at 365 nm for 2 h40 (about 10 J/cm²), irradiated under visible light for 2 h40 (I@visible) or left unirradiated. Papers were rinsed extensively with phosphate buffer enriched in salts and detergent. Grafting rate and activity rate were assessed.

As can be seen in FIG. 6, irradiation under visible light only slightly improves the grafting rate when compared to simple drying. Nevertheless, the corresponding activity rate appears to be slightly improved. Irradiation under visible light thus provides a better grafting than mere drying. Further, irradiation at 365 nm (more energetic) provides a better grafting than irradiation under visible light.

### Example 9: Influence of Ageing

OVA1 antibodies were poured onto CF1 cellulose or on nitrocellulose sheets. The impregnated CF1 papers were dried for concentrating the antibodies (S) or left undried. Dried impregnated papers were then irradiated (I) at 365 nm for 2 h40 (about 10 J/cm²). Papers were rinsed extensively with phosphate buffer enriched in salts and detergent. Grafting rate and activity rate were assessed. Nitrocellulose sheets were impregnated by the biomolecule solution and this system was left to incubate for 1 h at room temperature.

Immunochromatographic tests were performed immediately after assembling of strips. Other strips, prepared previously and stored in oven 7 days at 40° C. were used for assessing the effects of ageing.

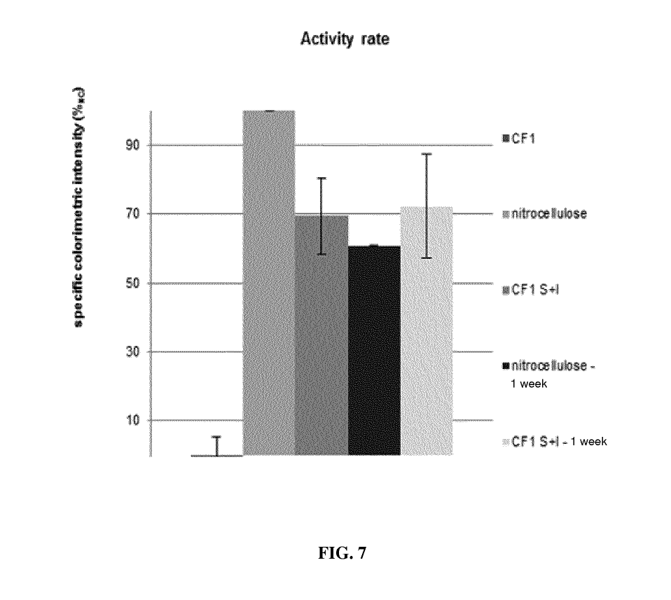

As can be seen in FIG. 7, ageing of nitrocellulose results in a decreased recognition of the grafted antibodies by the goat-anti-mouse antibody, as well as in a reduced biological activity. This phenomenon may be explained by the degradation of immobilized antibodies.

As regards cellulose, signal variability increases together with ageing. Recognition by goat-anti-mouse antibody is decreased, and may again result from the degradation of immobilized antibodies. Nevertheless, the observed decrease is less important than for nitrocellulose. Further, activity rate remains constant (when standard deviations are considered). These observations may be explained by the fact that antibodies grafted on cellulose through the method of the invention are less degraded at their binding site, or by the fact that they are “buried” in the paper, whereas antibodies are only displayed on the surface of nitrocellulose. Cellulose membranes according to the process of the present invention thus appear to be more resistant to ageing than nitrocellulose ones, and are therefore more suitable for use after storage.

### Example 10: Localized Immobilization of Labelled Antibodies

Photolithography consists in transferring an image displayed on a mask towards a substrate through photochemical or photoactivated reactions. The process according to the present invention now allows photolithography to be performed on cellulose sheets.

Probe antibodies labelled with colloidal gold were immobilized through a mask for allowing grafting of antibodies by photolithography to be observed directly, and for evaluating the signal/background ratio.

A solution of colloidal-gold-labelled goat-anti-mouse antibodies prepared accordingly to known methods (Khreich et al., Analytical Biochemistry 377 (2008) 182-188) was diluted three times, then poured onto a 2 cm² cellulose sheet (1×2 cm), at a rate of 20 μl/cm². Drying was intentionally omitted for avoiding important background levels resulting from the adsorption of gold particles. A mask was then placed on the antibody-impregnated membrane, and the system was irradiated for 80 minutes (about 5 J/cm²). After removal of the mask, samples were rinsed overnight with a phosphate buffer enriched in salts and detergent.

Gelatin (at a concentration of 1 mg/mL in potassium phosphate buffer 0.1M, pH 7.4) was immobilized according to the same procedure, and was used as negative control. The immobilization rate of labelled antibodies was measured by assessing the differences between the signal obtained with the paper coated with labelled antibodies, and the signal obtained with the gelatin-coated paper.

Results indicate that a selective photoimmobilization of the colloidal-gold-labelled antibody is observed according to the design of the used mask (data not shown).

### Example 11: Localized Immobilization of Simple Murine Antibodies

Simple murine antibodies were immobilized for ensuring that the selective photoimmobilization observed in example 10 was not resulting from the mere binding of colloidal gold particles. The antibodies (in solution at a concentration of 1 mg/mL in potassium phosphate buffer 0.1M, pH 7.4) were immobilized according to the general method used in previous examples. Membranes were then saturated with gelatin, and immobilized antibodies were detected with a colloidal-gold-labelled goat-anti-mouse antibody, similarly to the procedure previously implemented for evaluating the grafting rate.

Results indicate that the resulting photolithographic image matches with the selective immobilization of antibodies obtained by partial irradiation of the cellulose substrate (data not shown).

### Example 12: Immobilization of Labelled Antibodies

In order to assess the strength of grafting in the course of the photolithographic process, colloidal-gold-labelled antibodies were immobilized according to the general procedure implemented in previous examples. Following rinsing, colorimetric intensity was first measured. The antibody-grafted paper was then immersed in a solution of phosphate buffer enriched in salts and detergent and subjected to ultrasonic treatment for 20 minutes. Colorimetric intensity was measured again.

Results indicate that the colorimetric intensity measured after the ultrasonic treatment amounts to about 99% of the first intensity measured (data not shown). Considering that the observed signal decrease is comprised within the measuring error deviation, it can therefore be considered as non-significant. In conclusion, the grafting resulting from the process of the invention is thus very strong, if not covalent.

### Example 13: Immobilization of Various Antibodies

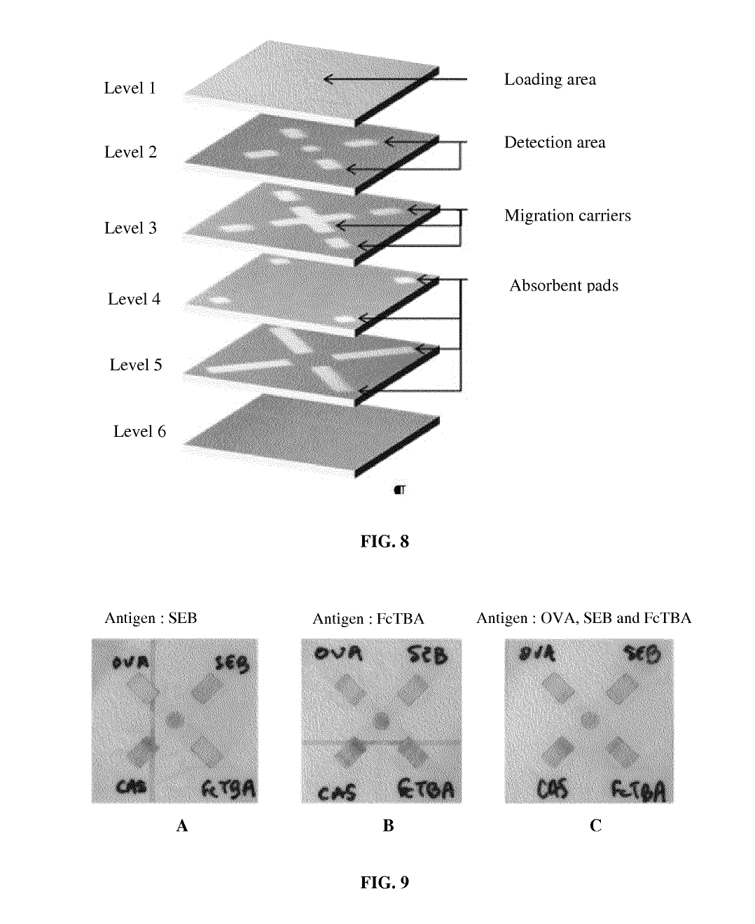

A multiplex system is generally composed of hydrophilic paper channels, delimited by hydrophobic plastic partition walls. The several levels of the system are stacked and connected by double-faced tape for forming a 3D fluidic system (see FIG. 8). The sample introduced at the top of the dispositive is divided in 4 aliquots, directed towards the 4 parallel assay areas of the dispositive. Papers grafted with different antibodies are introduced in the various assay areas, and thus allow different antigens to be detected within a same sample.

In the present experiment, the introduced sample was composed of mixed antigens and corresponding tracers. 3 out of the 4 assay areas were used for detecting antigens within the sample, while the last one was used as a control area for assessing the presence of tracers within the migrating system. The control area was thus grafted with goat-anti-mouse antibodies. The antigen used were ovalbumin (OVA), staphylococcus enterotoxin B (SEB) and a fragment of botulinum toxin A (Fc-TBA). The solid phase/antigen/tracer systems used were: anti-OVA antibody (OVA1)/OVA/colloidal-gold-labelled anti-OVA antibody (OVA35), anti-SEB antibody (SEB27)/SEB/colloidal-gold-labelled anti-SEB antibody (SEB26) and anti-Fc-TBA antibody (TBA11)/Fc-TBA/colloidal-gold-labelled anti-Fc-TBA antibody (TBA4). As described previously for measuring activity rates, antigen detection was performed by immunosandwich detection.

Paper channels and plastic position walls were cut using a laser plotter. Antibody grafted membranes were prepared similarly to the method previously described for preparing strips, i.e. through grafting, rinsing and saturating the membrane. Loading and migration areas were saturated with gelatin.

The various antibodies were therefore poured onto CF1 cellulose sheets, then concentrated by drying (S) of the impregnated paper. The resulting system was irradiated (I) at 365 nm for 2 h40 (about 10 J/cm²). Paper was extensively rinsed with phosphate buffer enriched in salts and detergent.

The tested solution comprised the 3 tracers diluted 10 times in the analysis buffer, as well as one or several antigens at a concentration of 1 μg/mL. 50 μL of the tested solution were introduced in the dispositive. The dispositive was then rinsed by the addition of 40 μL of the analysis buffer for reducing the unspecific signal and thereby allowing a better reading of the immunochromatographic results.

As can be seen in FIGS. 9A, 9B and 9C, for each assay the specific area corresponding to the introduced antigen was colored. Nevertheless, the coloration is slightly weaker when the tested solution contains all the antigens.

### Example 14: Localized Immobilization of Probe Antibodies

Photo-patterning (photolithography) consists in transferring an image displayed on a mask towards a substrate through photochemical or photoactivated reactions. This is the fastest and most easily undertaken process ensuring the localization of species onto a flat carrier according to a well-defined and reproducible pattern. This process was therefore combined to the above disclosed chemical-free photografting procedure in order to easily and rapidly localize antibodies onto cellulose sheets.

Probe antibodies labeled with colloidal gold were immobilized through a mask in order to directly observe the photo-patterned immobilization of antibodies, and to evaluate the signal/background ratio (FIG. 12). A selective photoimmobilization of the colloidal-gold-labeled antibody was observed according to the design of the used mask.

This confirms the immobilization process to be photo-controlled. The signal/background ratio is estimated to be around 140%. Though it is a rather positive result, the high background colorimetric intensity also indicates that lots of antibodies are wasted in this process. That stems from the subtractive nature of the photo-patterning process. An additive process, such as inkjet printing, was thus tested.

### Example 15: From Classical Automatic Dispensing to Inkjet Printing of Antibodies

Since automatic dispensing with BioDot-like systems (Khreich et al., Anal. Biochem. 2008, 377, 182-188) is the most frequently used method for antibody dispensing onto immunoassay membranes, the inkjet printing approach was first compared to the latter. That comparison aimed to validate the printing method for its use in the development of immunoassay devices.

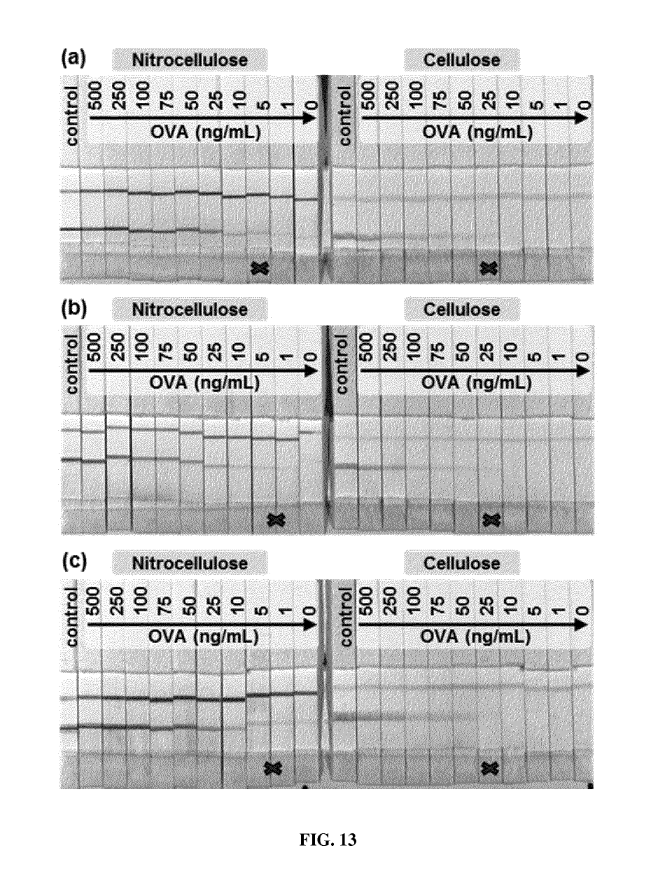

Printings made of 1 and 5 layers were therefore compared to the single line deposit from the automatic dispenser (FIG. 13). After antibody solutions had been dispensed onto the substrates, the antibodies were either adsorbed onto nitrocellulose or photoimmobilized onto cellulose. First, their immobilization was confirmed by revelation with gold-labeled goat anti-mouse tracer (see control strips in FIG. 13). Then, their biological activity was put to the test by exposition to OVA antigen and simultaneously revealed by gold-labeled murine anti-OVA tracer (sandwich immunoassay) (see OVA strips in FIG. 13). Each test was performed in triplicate.

As a result, the sets of strips obtained with BioDot dispensing method and with 5-layer inkjet printing are visually almost identical. Their coloring is quite strong, while the coloring resulting from 1-layer inkjet printing is obviously weaker. However, this weakness does not seem to lower its performances in terms of visual detection limit (VDL) as further detailed. This same set of strip actually displays slightly thinner and more precise test and control lines than the others, although they are all well-defined, thin and precise. With regard to biological activity, dilutive effect is clearly perceptible. Nevertheless, photographs reveal that the negative control (OVA at 0 ng mL⁻¹) for nitrocellulose is slightly colored. This raises the issue of false positive results that can be observed with nitrocellulose immunoassay membranes. This issue does not arise with cellulose, most probably because of lower sensitivity. Considering that, the membranes' VDL were appraised as follows: (i) 5 ng mL⁻¹ for nitrocellulose and 25 ng mL⁻¹ for cellulose with BioDot dispensing method (FIG. 13a); (ii) 1 to 5 ng mL⁻¹ for nitrocellulose and 25 ng mL⁻¹ for cellulose with 1-layer inkjet printing (FIG. 13b); and (iii) 1 to 5 ng mL⁻¹ for nitrocellulose and 25 ng mL⁻¹ for cellulose with 5-layer inkjet printing (FIG. 13c).

Each material VDL was therefore identical regardless the dispensing method or the number of layers. Thus, the printing process was indeed proved to be as efficient as the usual automatic dispensing, and therefore totally legitimate regarding its use in the development of immunoassay devices. Moreover, the printing method has the advantage of saving the quite expensive biomolecules dispensed because of the rather low ejected volume. Though an exact ejected volume could not be measured, the maximum dispensed volume was calculated based on the printer features (nominal drop volume, drop spacing and tension). For the selected pattern (a straight line of 600 μm width), the printer was estimated to deliver 0.27 μL cm⁻¹ of antibody solution per layer. A maximum of 0.27 μL cm⁻¹ of antibody solution was thus dispensed with 1-layer inkjet printing (FIG. 13b), a maximum of 1.35 μL cm⁻¹ with 5-layer inkjet printing (FIG. 13c), and exactly 1 μL cm⁻¹ with BioDot dispensing method (FIG. 13a). Since a 1-layer printing is efficient enough to determine the VDL, the consumed amount of antibodies is therefore nearly a quarter of the amount consumed with a classical automatic dispenser. Another advantage of printing over classical automatic dispensing is the freedom in design of the printed pattern (see below) while the usual automatic dispenser only allows drawing straight lines of rather undefined width.

Regarding the evaluation of the immobilization procedure, photoimmobilization onto cellulose led to VDL results in the same order of magnitude as the values obtained with adsorption onto nitrocellulose. However, cellulose performances appeared slightly lower than nitrocellulose's (VDL_{cellulose}=5 VDL_{nitrocellulose}). Beyond procedure, this phenomenon might stem from the many differences both chemical and physical between the two substrates. These differences were characterized and cellulose pretreatments were tested for trying to compensate for them.

### Example 16: Inkjet Printing of Antibodies onto Various Substrates

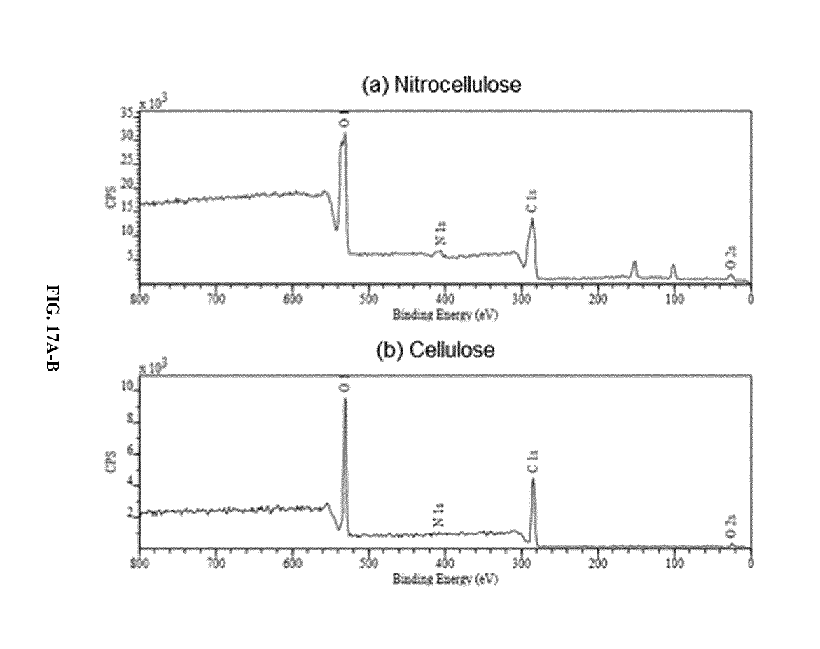

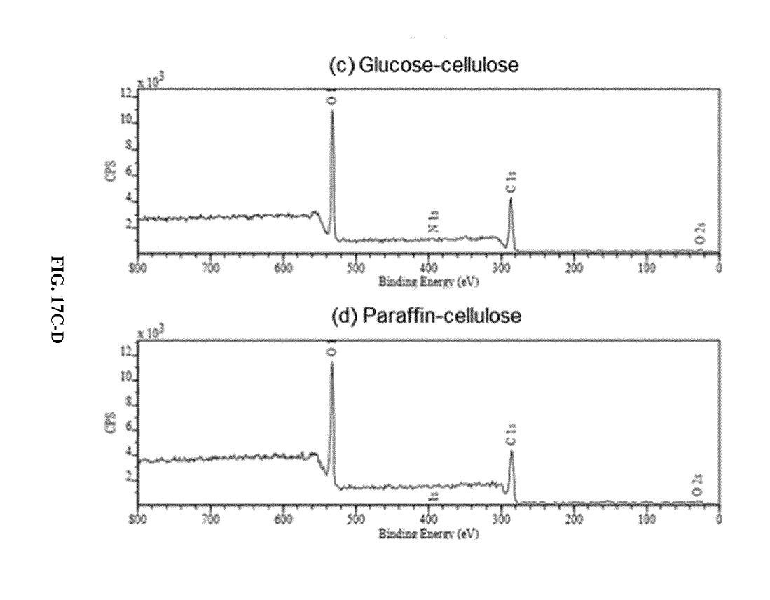

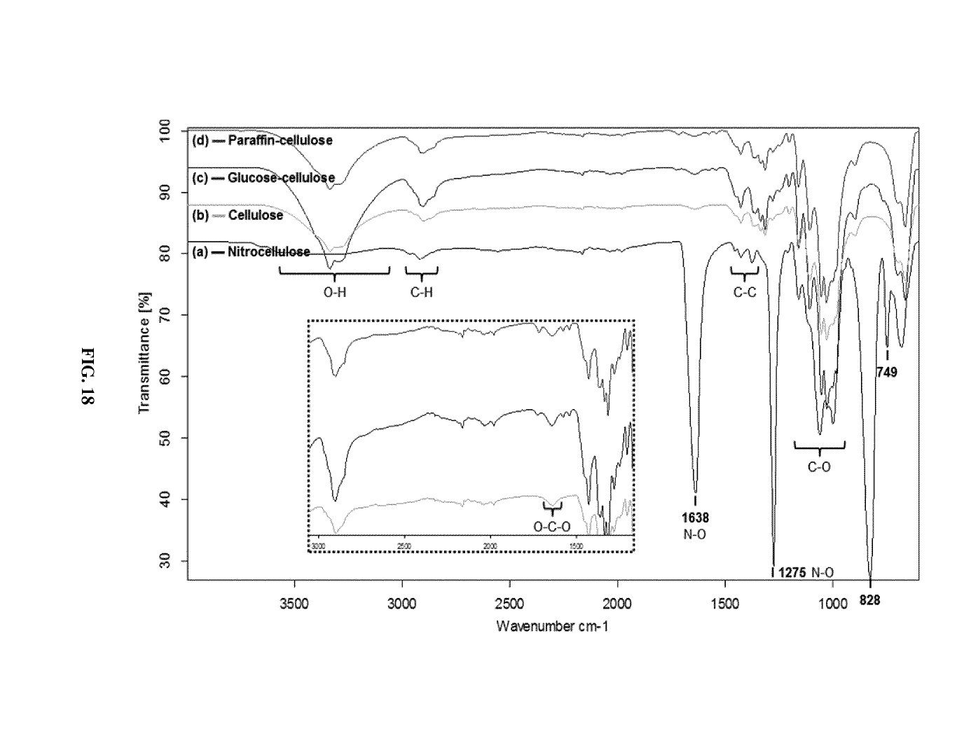

Beyond the obvious chemical difference in molecular structure, the main physical difference between nitrocellulose and cellulose substrates lies in their porosity (about 5 μm and 11 μm surface pore size, respectively) and sheet thickness (20 μm and 176 μm thick, respectively). Since cellulose sheets with same porosity and thickness than nitrocellulose were not commercially available, compensative cellulose pretreatments were performed by filling cellulose pores. The filling substance should be inert regarding antibody immobilization process and further immunoassays. Two components were initially selected: glucose and paraffin. Glucose is the molecular repeating unit in cellulose macromolecule (see FIGS. 16a and b) and therefore was not expected to disturb the immobilization process or further use of the membrane. In addition, its high water solubility (180 mg mL⁻¹) would permit to easily remove it during post-irradiation washing step. Paraffin, a mixture of linear alkanes (see FIG. 16b), is well known for its unreactive nature (Noh et al. Anal. Chem. 2010, 82, 4181-4187). Unlike glucose, it is insoluble in water and therefore would stick into the fibers after the washing step and during further immunoassays.

Antibody solutions were printed onto the raw (nitrocellulose and cellulose) and pretreated (glucose-cellulose and paraffin-cellulose) substrates. Though 1 layer would have been enough, 5 layers were actually printed in order to get strong color intensity. Antibodies were then adsorbed onto nitrocellulose substrate and photoimmobilized onto cellulose substrates (cellulose, glucose-cellulose and paraffin-cellulose). Surface morphological structure and chemical composition of both raw and pretreated substrates were analyzed prior to printing and afterwards. Printed antibody solutions were characterized as well. Finally, lateral flow immunoassays (LFIAs) ensured the ultimate characterization by evaluating the biological activity and visual detection limit of the various membranes.

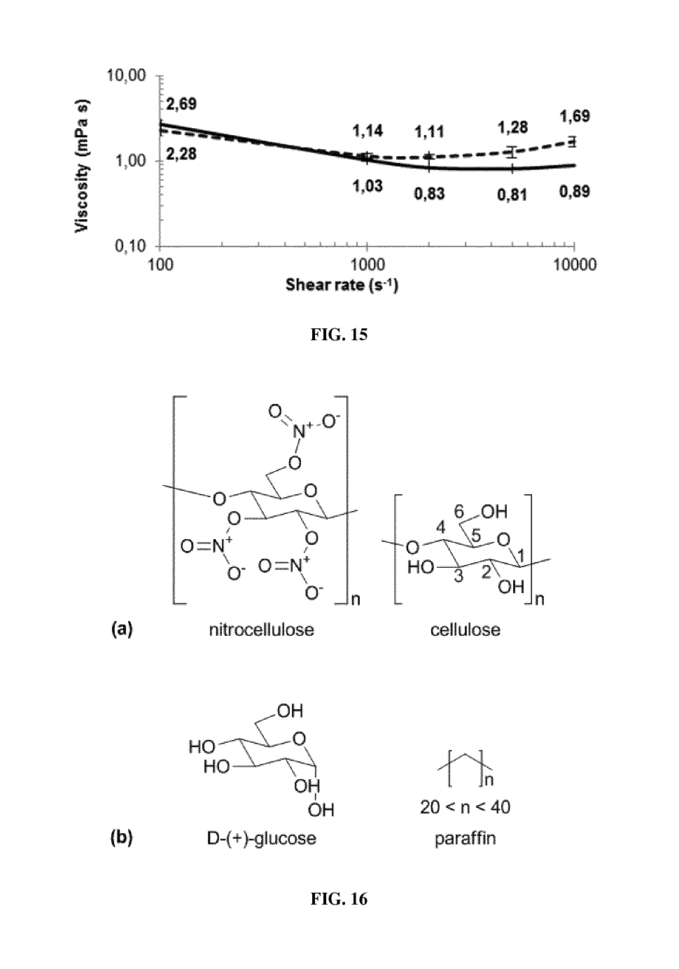

The viscosity of both test line and control line antibody solutions was measured (FIG. 15). As reminded above, test line ink consisted of murine anti-OVA monoclonal antibodies and control line ink of goat anti-mouse polyclonal antibodies. According to FIG. 15, control line ink viscosity varies from 2.28 to 1.69 mPa s when shear rate increases from 100 to 10 000 s⁻¹. A slight increase of viscosity is observed at shear rates higher than 2 000 s⁻¹. The control line solution is thus dilatant. Test line ink viscosity varies from 2.69 to 0.89 mPa s for the same shear rate ranges. The test line solution has a shear thinning behavior.

Equation 1 below is the expression of the shear rate as a function of gap and printing speed.

$$\dot{γ} = \frac{v}{h} , \qquad Equation 1$$

wherein \dot{γ} is the shear rate (s⁻¹), v is the velocity (m s⁻¹) and h is the gap (m).

When shear rate varies from 100 to 10 000 s⁻¹, speed varies from 0.01 to 1 m s⁻¹ for a gap of 100 μm (1×10⁻⁴ m). Depending on ink viscosity and printing voltage, jetting speed thus varies from 0.1 to 25 m s⁻¹ (Denneulin et al., Carbon N.Y., 2011, 49, 2603-2614). Hence, high shear rates larger than 10 000 s and exceeding the rheometer measuring limits may be estimated.

Ideally, an inkjet printing ink must be Newtonian with a constant viscosity (1-10 mPa s) at varying shear rates (Blayo et al., sOc-EUSAI '05, ACM Press: New York, N.Y., USA, 2005, pp. 27-30). Though not Newtonian, biomolecule solutions are inkjet printable because of their low viscosities (<2 mPa s).

### Example 17: Surface Chemical Treatment Analysis

The outer surface layers of paper substrates were analyzed by surface chemical analysis such as XPS and ATR-FTIR, thereby displaying the aforementioned bulk molecular structures.

XPS allows the identification of elements within 10 nm deep subsurface layers (Johansson et al., Surf. Interface Anal. 2004, 36, 1018-1022). All papers are mainly composed of carbon and oxygen and therefore the XPS signal for these two elements is quite strong on every spectrum shown. FIG. 17 displays O 1s orbital Binding Energy at 532 eV±0.35 eV, O 2s orbital Binding Energy at 24 eV±0.35 eV and C 1s orbital Binding Energy at 284 eV±0.35 eV) (Johansson, et Campbell, J. M. Reproducible XPS on biopolymers: cellulose studies. Surf. Interface Anal. 2004, 36, 1018-1022). Another peak at 405±0.35 eV is noticeable onto nitrocellulose spectrum which is attributable to N 1s orbital.

According to its layout, ATR-FTIR allows the identification of chemical bonds within 2 μm deep subsurface layers (PIKE technologies MIRacle ATR, Product data sheet, Madison, Wis., USA, 2014). All papers are mainly composed of a cellulosic backbone and therefore the IR signals for its typical bond vibrations are shared by every spectrum shown. FIG. 18 displays these common bands attributable to O—H, C—H, C—C, C—O and O—C—O stretching vibrations. Besides, nitrocellulose manifests additional peaks (1638±5 cm⁻¹ and 1275±5 cm⁻¹) attributable to N—O stretching vibrations.

### Example 18: Analysis of Surface Morphological Structure

Beyond the chemical differences in molecular structure, the main difference between nitrocellulose and cellulose substrates lies in their surface physical structure. Thus, topological analysis was conducted in order to quantify the surface morphological structure by measuring its roughness (Ra). SEM imaging allowed visualizing surface morphology and microstructure of the unprinted substrates.

The porosity of the treated cellulose sheets was analyzed by Scanning Electron Microscopy (SEM). Results are presented in FIG. 17 A (untreated CF1 sheet), and 17 B) (CF1 sheet obtained after treatment).

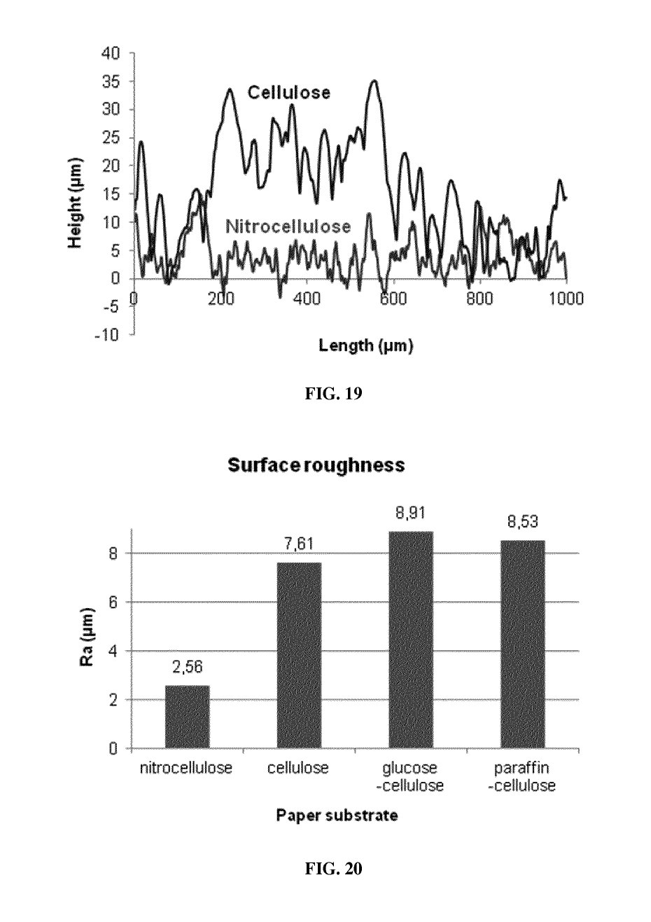

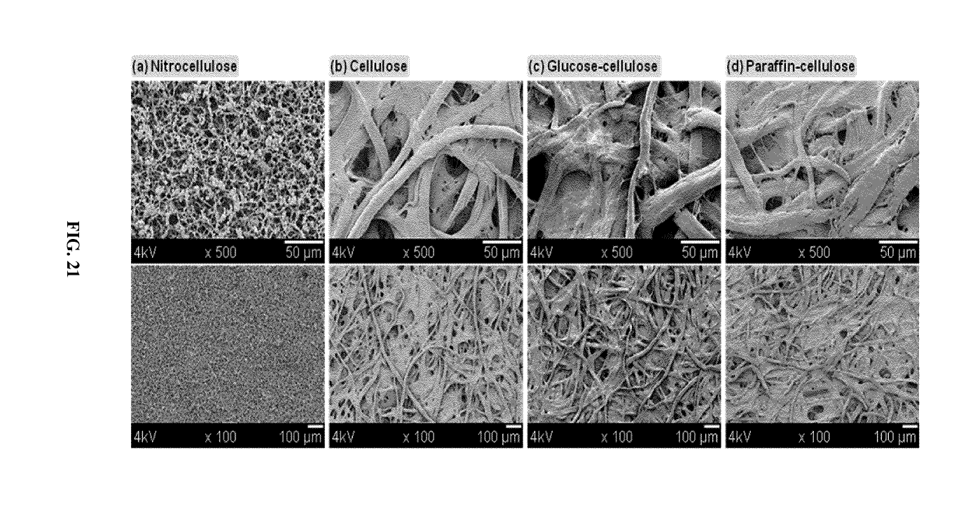

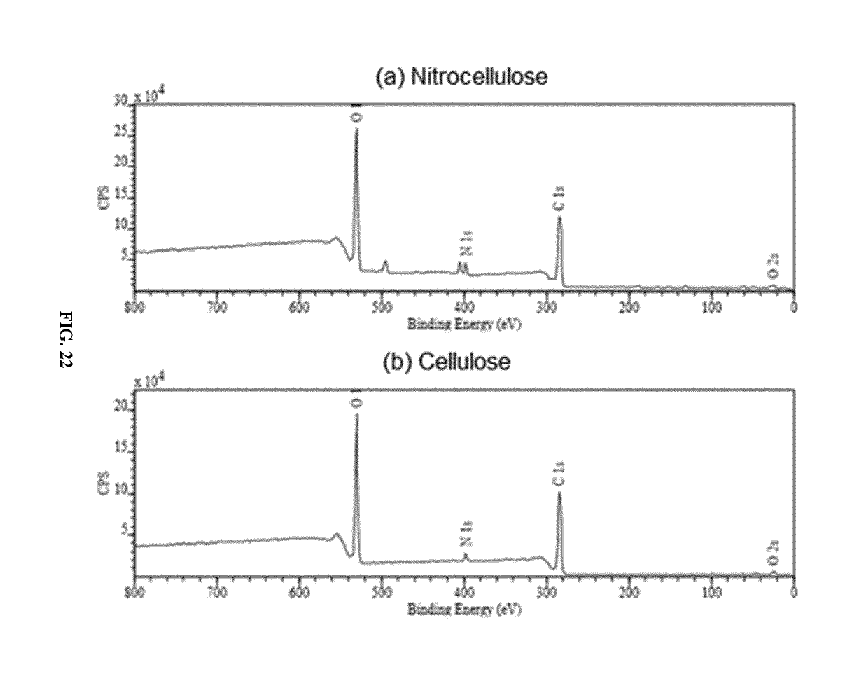

Line profiles of unprinted paper substrates (FIG. 19) revealed that nitrocellulose surface is more homogeneous, smoother and has fewer and narrower pores compared to cellulose-based paper surfaces. Since profiles of the three cellulose-based papers were quite similar, only cellulose profile is displayed on FIG. 19. Surface roughness (Ra) values (FIG. 20) confirmed that nitrocellulose is way smoother than cellulose-based papers. Pores size and arrangement pictured by SEM imaging (FIG. 21) also corroborated the previous statements. SEM micrographs and roughness profiles predict that with the same ejected volume of antibodies, thicker and better resolution patterns will be printed on nitrocellulose. Thus, lower visual detection limits are expected to be reached with nitrocellulose membranes. This was supported by Määttänen et al. (Määttänen, A. et al., Colloids Surfaces A Physicochem. Eng. Asp. 2010, 367, 76-84) who demonstrated that wetting rate reduces with surface roughness increase. Besides, they explained that ink is quickly and completely absorbed into the depth of porous surfaces, thus leaving less ink deposit onto the substrate surface.

According to SEM imaging (FIG. 21), glucose treatment seemed to barely affect cellulose surface aspect. On the other hand, when paraffin treatment was performed, fewer pores were observed onto the surface. Regarding surface roughness (FIG. 20), an increase was displayed by both glucose and paraffin treatments.

### Example 19: Surface Chemical Analysis of the Printed Substrates

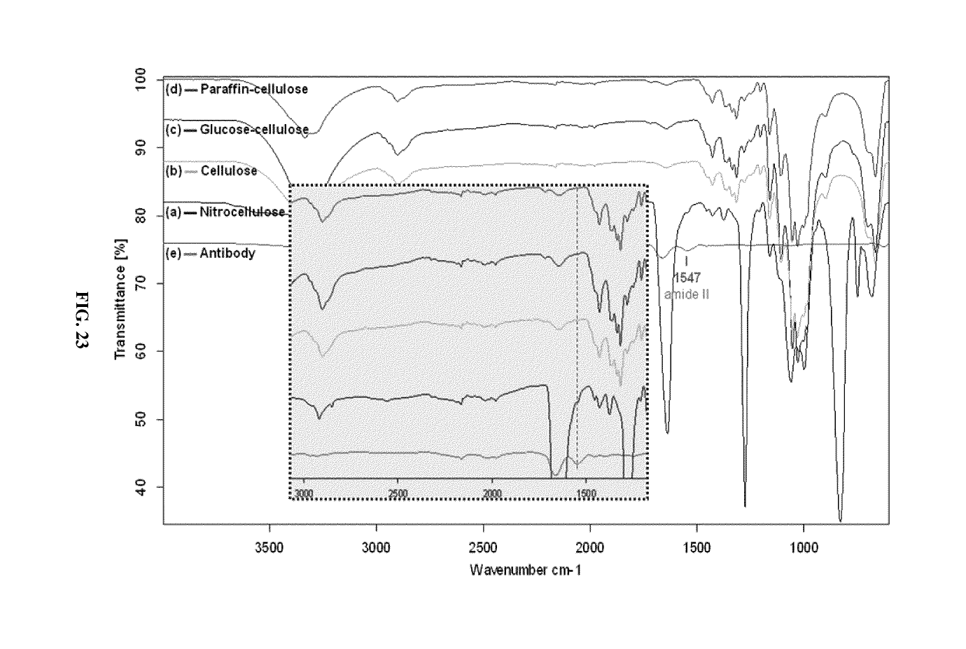

After antibody had been printed onto the various paper substrates, their outer surface layers were analyzed anew in order to detect any change stemming from the biomolecules. The XPS signal from carbon and oxygen was still quite strong on every spectrum shown (FIG. 22). Additional peaks at 397.5±0.35 eV have come out onto all the spectra which are attributable to N 1s orbital from antibody molecules. Since spectra of the three cellulose-based papers were quite similar, only cellulose spectrum is displayed on FIG. 22.

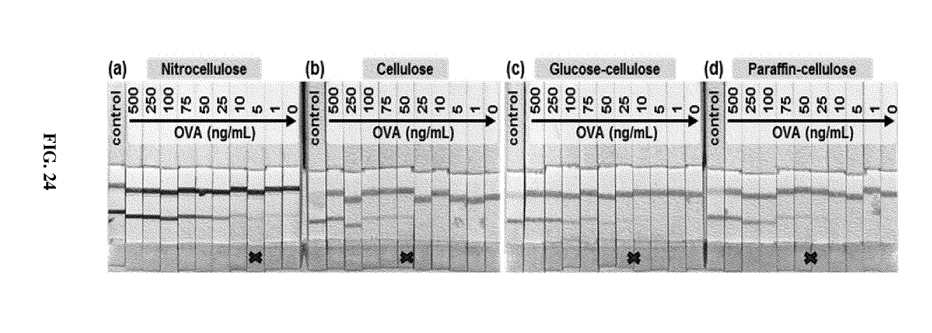

With regard to IR analysis, the intense spectra from initial substrates hid most of the characteristic bands pointing out the immobilized antibodies (FIG. 23). Therefore, the amide bands specific to proteins were barely perceivable. Only amide II at 1547±5 cm⁻¹ could be clearly identified onto nitrocellulose substrates.

### Surface Morphological Structure

After antibody had been printed onto the various paper substrates, their surface morphology and microstructure were visualized anew (not shown) by SEM imaging in order to detect any change stemming from the biomolecules. Unfortunately, the microscope resolution was not high enough to enable a direct visualization of antibody deposit. However, a thin new layer seemed to have appeared on cellulose-based substrates when comparing to FIG. 21.

### Lateral Flow Immunoassays (LFIAs)

Antibody solutions were printed onto the raw (nitrocellulose and cellulose) and pretreated (glucose-cellulose and paraffin-cellulose) substrates. 5 layers were printed in order to get strong color intensity. Antibodies were then adsorbed onto nitrocellulose substrate and photoimmobilized onto cellulose substrates (cellulose, glucose-cellulose and paraffin-cellulose). Lateral flow immunoassays (LFIAs) evaluated the biological activity of the printed antibodies and the visual detection limit of the various bioactive membranes, thereby allowing characterization of the various substrates in terms of biosensing performances. First, the immobilization ability of the various membranes was confirmed by revelation with gold-labeled goat anti-mouse tracer (see control strips in FIG. 24). Then, their biological activity was assessed by exposition to OVA antigen and revealed by gold-labeled murine anti-OVA tracer (sandwich immunoassay) (see OVA strips in FIG. 24). Each test was performed in triplicate.

Though antibodies were barely perceivable with the various surface analysis performed (XPS, IR or SEM), they were well visible after either revelation with goat anti-mouse tracer (control strips) or bioactivity assessing immunosandwich (OVA strips). With regard to biological activity, few aforementioned results (see above) remain. Dilutive effect was still clearly perceptible. There was still a false positive result with nitrocellulose that compelled to appraise its VDL at 5 ng mL⁻¹ (FIG. 24a). The other VDLs were 50 ng mL⁻¹ for cellulose (FIG. 24b), 10 to 25 ng mL⁻¹ for glucose-cellulose (FIG. 24c), and 25 to 50 ng mL⁻¹ for paraffin cellulose (FIG. 24d). While nitrocellulose's VDL is still the same as described above, cellulose's VDL is now higher. Since all test lines coloring seemed weaker than in FIG. 13c, this inter-assay variability could originate from tracer variability due to the use of another batch of colloidal gold. On another hand, the intra-assay comparison of the different substrates revealed that both glucose and paraffin enrichment slightly improved cellulose performances although they were still lower than nitrocellulose's. Besides, glucose-cellulose appeared to be the most sensitive cellulose-based substrate. This could be explained by a slight decrease in surface porosity, as expected.

### Example 20: Inkjet Printing of Complex Designs

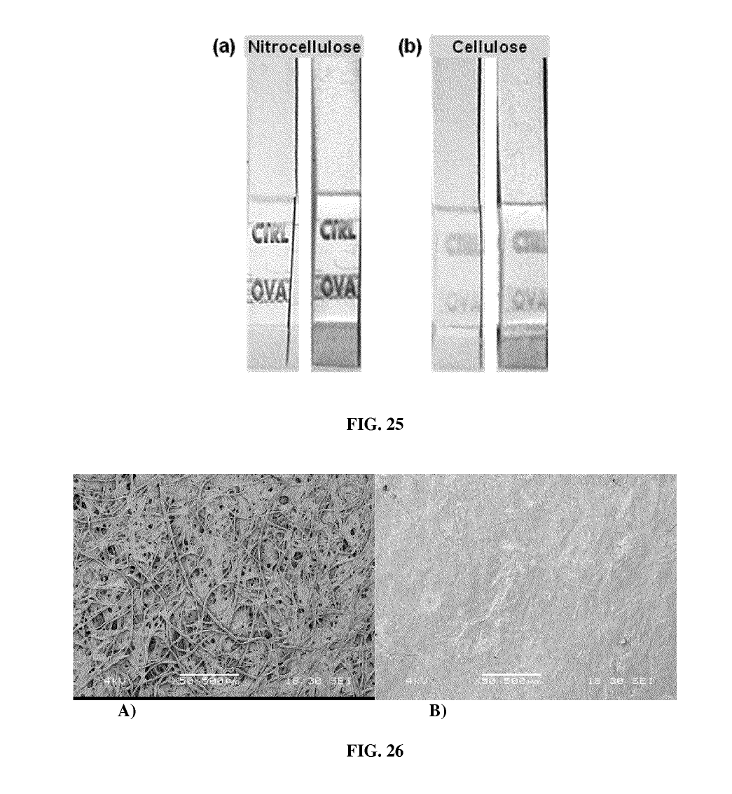

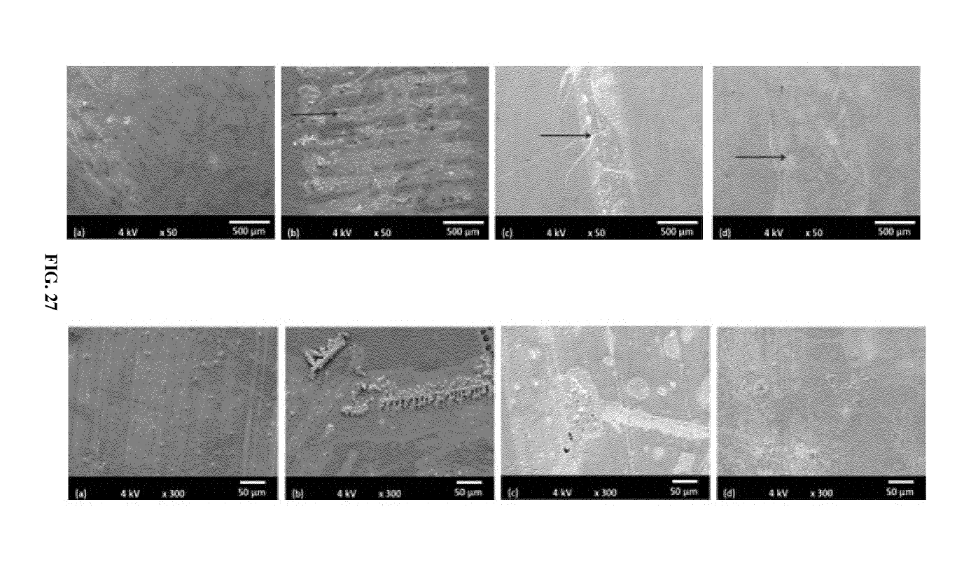

As previously mentioned, one advantage of inkjet printing dispensing method is the freedom in design of the printed pattern. This advantage was illustrated here by printing antibodies according to their nature and function, thereby making the user manual not so useful anymore. Since bottom line was dedicated to capture OVA antigen, murine anti-OVA monoclonal antibodies printing drew the abbreviation OVA. Similarly, anti-mouse antibodies were printed on the top line according CTRL abbreviation as the top line aimed to control the smooth progress of the immunoassay. After antibody solutions had been dispensed onto the substrates (1-layer inkjet printing), the antibodies were either adsorbed onto nitrocellulose or photoimmobilized onto cellulose. Their biological activity was put to the test by exposition to OVA antigen (500 ng mL⁻¹) and simultaneously revealed by gold-labeled murine anti-OVA tracer (FIG. 25). Colors observed, along with their intensities, were consistent with previous results (see above). Finally, as expected, the drawn patterns allowed direct reading of the test results. This process therefore enables to doubly check the nature of the target antigen (on the box and on the strip), thereby avoiding ambiguousness when box label is partly erased. Firstly, this can permit to save valuable assay devices in remote areas in the developing world. In addition, this double-check can be a huge asset in developed countries in emergency situations, in emergency rooms or in military settings, where the result of the assay impacts on people's lives.

The herein exemplified method thus constitutes a fast, simple, cost-saving and environmentally friendly method for strong and precisely localized immobilization of antibodies onto paper. Further, the combination of inkjet printing of biomolecules with a chemical-free photografting procedure according to the invention together enable to easily, rapidly and permanently immobilize antibodies onto cellulose-based papers according to any pattern desired. The inkjet printing dispensing method has the great advantage of saving the expensive biomolecules. The photografting procedure has the one of being harmless to chemical-sensitive biomolecules.

### Example 21: Assays Conducted on Additional Cellulose Pretreated Sheets

Additional cellulose pretreated sheets have been prepared and studied. The resulting membranes were shown to be capable of challenging nitrocellulose performances. Cellulose-based pretreated sheets performances nevertheless appeared slightly lower than nitrocellulose's though. As discussed above, this phenomenon presumably stems from the physical differences, such as surface porosity variation, between nitrocellulose and cellulose substrates.

The photografting of pretreated cellulose, and more generally of substantially non-porous carriers meets the need for sensing devices development to rapidly, robustly and abundantly immobilize biomolecules onto substrates or carriers according to complex patterns and at low cost. The expounded process thus provides a powerful tool for immobilizing chemical-sensitive proteins according to complex patterns and onto various substantially non-porous carriers, including cellulose-based paper sheets.

Several additional filling substances were tested in combination with cellulose sheets:
Poly(acrylic acid) sodium salt, 35% in H₂0 Mw 60.000 (PolySciences Inc);
Poly(acrylic acid) sodium salt, 35% in H₂0 Mw 250.000 (Sigma Aldrich);
Poly(acrylic acid) powder Mw 1.000.000 (PolySciences Inc);
Poly(styrenesulfonic acid-Maleic anhydride), 3:1, Low Mn (Polysciences Inc);
Poly(vinylphosphonic acid), 30% in H₂0;
PolyStyrene solid, Mw 192.000 (Sigma Aldrich); and
Poly(ethyleneglycol) Mw 950-1.050 (Sigma Aldrich).

### Example 21.a): Pretreatment of Cellulose Sheets with a Solution of Poly(Acrylic Acid) Sodium Salt 35% (Mw 60.000)

A 35% w/v solution of Poly(acrylic acid) sodium salt (Mw 60.000 (PolySciences Inc)) was solubilized in H₂0 and filtered as disclosed above on XEROX Premier® or on Whatman CF1® cellulose sheets. Filtering was stopped when the polymer solution became unable to flow through the cellulose sheet. The resulting sheets were then dried at room temperature for 48 hours.

### Example 21.b): Pretreatment of Cellulose Sheets with a Solution of Poly(Acrylic Acid) Sodium Salt 35% (Mw 250.000)

A 35% w/v solution of Poly(acrylic acid) sodium salt (250.000 (Sigma Aldrich)) was solubilized in H₂0 and filtered as disclosed above on XEROX Premier® or on Whatman CF1® cellulose sheets. Filtering was stopped when the polymer solution became unable to flow through the cellulose sheet. The resulting sheets were then dried at room temperature for 48 hours.

### Example 21.c): Pretreatment of Cellulose Sheets with a Solution of PolyStyrene

A solution of PolyStyrene (Mw 192.000 (Sigma Aldrich)) was prepared by dissolving 3 g of polystyrene in 10 mL pure acetone (Sigma Aldrich). The supernatant only was collected, then filtered on XEROX Premier® or on Whatman CF1® cellulose sheets. Filtering was stopped when the polymer solution became unable to flow through the cellulose sheet. The resulting sheets were then dried at room temperature for 48 hours.

### Example 21.d): Pretreatment of Cellulose Sheets with a Solution of Poly(Acrylic Acid) (Mw 1.000.000)

A solution of Poly(acrylic acid) (Mw 1.000.000 (PolySciences Inc)) was prepared by dissolving 200 mg of polyacrylic acid in 10 mL Milli-Q H₂0. The solution was then filtered on XEROX Premier® or on Whatman CF1® cellulose sheets. Filtering was stopped when the polymer solution became unable to flow through the cellulose sheet. The resulting sheets were then dried at room temperature for 48 hours.

### Example 21.e): Pretreatment of Cellulose Sheets with a Solution of Poly(Acrylic Acid) (Mw 250.000), Poly(Vinylphosphonic Acid), Poly(Styrenesulfonic Acid-Maleic Anhydride, and Low Mn

A solution containing i) 2 mL of a 35% w/v Poly(acrylic acid) sodium salt solution (Mw 250.000 (Sigma Aldrich)) in H₂0, ii) 2 mL of a 30% Poly(vinylphosphonic acid) solution, in H₂0, and iii) 15 mL of Milli-Q H₂0 containing 1 g of Poly(styrenesulfonic acid-Maleic anhydride), Low Mn (Polysciences Inc) 3:1, and 1.5 g of Poly(ethyleneglycol) Mw 950-1.050 (Sigma Aldrich) was filtered on XEROX Premier® or on Whatman CF1® cellulose sheets. Filtering was stopped when the polymer solution became unable to flow through the cellulose sheet. The resulting sheets were then dried at room temperature for 48 hours.

### Example 21.f): Pretreatment of Cellulose Sheets with a Solution of Poly(Acrylic Acid) (Mw 250.000) then by a Solution of PolyStyrene

A 35% solution of Poly(acrylic acid) sodium salt (Mw 250.000 (Sigma Aldrich)) in H₂0 was filtered on XEROX Premier® or on Whatman CF1® cellulose sheets. The resulting sheets were then dried at room temperature for 24 hours.

A solution of PolyStyrene (Mw 192.000 (Sigma Aldrich)) was prepared by dissolving 3 g of polystyrene in 10 mL pure acetone (Sigma Aldrich). The supernatant only was collected, then filtered on the dried cellulose sheets. Filtering was stopped when the polymer solution became unable to flow through the cellulose sheet. The resulting sheets were then dried at room temperature for 48 hours.

### Example 21.g): Pretreatment of Cellulose Sheets with a Solution of Poly(Acrylic Acid) (Mw 250.000) then by a Solution of PolyStyrene

A 35% solution of Poly(acrylic acid) sodium salt (Mw 250.000 (Sigma Aldrich)) in H₂0 was filtered as described previously on XEROX Premier® or on Whatman CF1® cellulose sheets. Filtering was stopped and the cellulose sheets were dried at room temperature for 48 hours. The supernatant of a solution of PolyStyrene (Mw 192.000 (Sigma Aldrich)) prepared by dissolving 3 g of polystyrene in 10 mL pure toluene (Sigma Aldrich) was then filtered on the same cellulose sheets. Filtering was stopped when the polymer solution became unable to flow through the cellulose sheet. The resulting sheets were then dried at 40° C. for 48 hours.

### Example 22: Photochemical Grafting of Proteins on Polyethyle Terephthalate (PET)

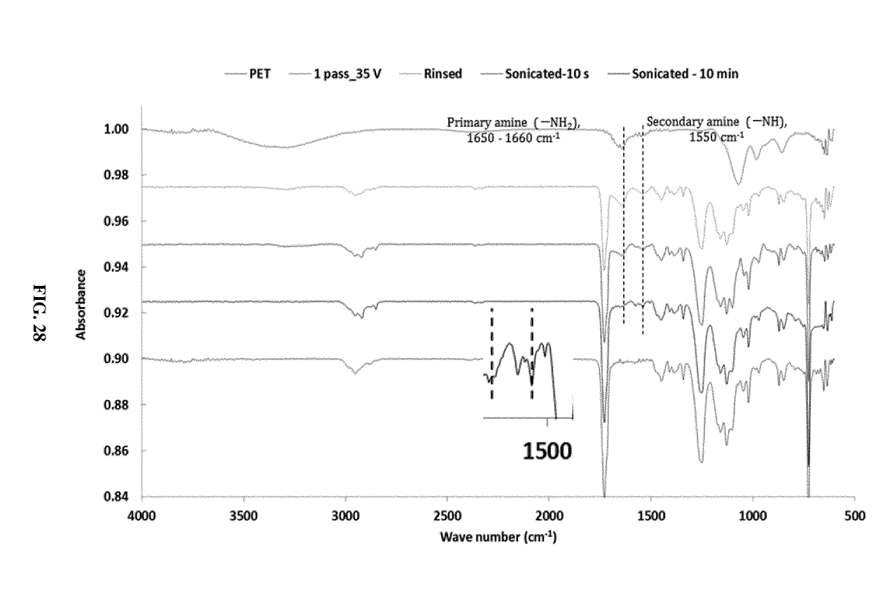

A solution of monoclonal antibody anti-OVA, at a concentration of 1 mg/ml, was printed onto a carrier of polyethylene terephthalate (PET) of a thickness of 100 μm, with an inkjet printer Dimatix Material Printer DMP-2800, having a distance printing head/carrier of 0.75 mm. The printed carrier was dried in an oven, at standard atmospheric pressure, at 37-40° C., then irradiations were conducted at room temperature under a 365 nm light, for 15 minutes. The grafted surface was then subjected to washing with ultrasound, then observed by SEM imaging. Results are displayed on FIG. 27A) and B). As could be seen in FIG. 27, trace amounts of proteins can still be observed after washing with ultrasound. Further, IR analysis of the grafted PET surface was conducted before and after washing with water and ultrasound. Results are displayed in FIG. 28. Peaks corresponding to primary amines, —NH₂ (around 1660 cm⁻¹), and to secondary amines, —NH (around 1550 cm⁻¹), can still be observed even after washing with water and ultrasound.

### Materials and Methods

### Materials

### Proteins

Proteins (ovalbumin (OVA), Bovine Serum Albumin (BSA) and porcine skin gelatin), as well as chemical products for preparing buffers, colloidal gold solution, and substrates pretreatment mixtures were obtained from Sigma-Aldrich (St Louis, Mo., USA). Water used in all experiments was purified by the Milli-Q system (Millipore, Brussels, Belgium).

Monoclonal murine antibodies (murine mAbs) were produced at LERI (CEA, Saclay, France) as described by Khreich et al. (Khreich et al., Toxicon 2009, 53, 551-559). Goat anti-mouse antibodies (IgG+IgM (H+L)) were purchased from Jackson ImmunoResearch (West Grove, Pa., USA).

### Initial Papers and Substrates

Papers used for preparing the immunoassay membranes were CF1 and Chr1 celluloses, as well as AE 98 Fast nitrocellulose from Whatman (Maidstone, Kent, UK), and printing paper Xerox premier 80 (Ref. 3R91720, Xerox, Norwalk, Conn., USA). Papers were used as received.

Cellulose is a natural biopolymer made up of glucose units (FIG. 16a). It is the simplest polysaccharide since it is composed of a unique monomer (glucose) which binds to its neighbors by a unique type of linkage (β-1,4 glycosidic bond resulting in acetal function). According to its molecular structure, hydroxyl groups in glucose units are responsible for cellulose chemical activity (Roy et al., Chem. Soc. Rev. 2009, 38, 2046-2064). However, this group cannot directly interact with proteins, what usually makes cellulose activation or functionalization necessary in order to covalently bind to proteins of interest.

Nitrocellulose (also named cellulose nitrate) is the most important cellulose derivative. Biomolecules strongly adsorb to nitrocellulose through a combination of electrostatic, hydrogen, and hydrophobic interactions involving the nitro functions. It is therefore the reference material for performing lateral flow immunoassay (LFIA) (Posthuma-Trumpie et al., Anal. Bioanal. Chem. 2009, 393, 569-582; Ngom et al., Anal. Bioanal. Chem. 2010, 397, 1113-1135; Fridley et al., MRS Bull. 2013, 38, 326-330). Cellulose nitrate is formed by esterification of hydroxyl groups from cellulose (primary or secondary) with nitric acid in the presence of sulfuric acid, phosphoric acid or acetic acid (see FIG. 16a) (Roy et al., Chem. Soc. Rev. 2009, 38, 2046-2064).

Polyethylene terephthalate (PET) was obtained from 3M or HP.

### Pretreated Papers and Substrates

Cellulose pretreatments were performed with additive molecules (in particular on CF1 cellulose and Xerox premier cellulose), which did not change the native chemical structure of cellulose. Additive substances were adsorbed onto cellulose such as to partially fill its pores. Additives used comprise glucose, paraffin, Poly(acrylic acid), Poly(styrenesulfonic acid-Maleic anhydride), Poly(vinylphosphonic acid), PolyStyrene and Poly(ethyleneglycol). While glucose is the molecular repeating unit in cellulose macromolecule, paraffin is a mixture of linear alkanes (see FIG. 16b).

Glucose-cellulose was prepared by dipping a CF1 cellulose sheet in a 100 mg·mL⁻¹ aqueous solution of D-(+)-glucose overnight at 4° C., and then drying it at 37° C. in a ventilated oven for 1 hour.

Paraffin-cellulose was prepared by dipping a cellulose sheet in a 10 mg·mL⁻¹ hot aqueous suspension of paraffin for 1 hour, and then drying it at 37° C. in a ventilated oven for 1 hour. The temperature of the aqueous solution needed to be above 60° C. for paraffin to melt and mix with water.

Other pretreated celluloses were prepared as follows: a sheet of cellulose was placed on a sintered glass or on a Millipore filtration system (suitable for filtering on polymer membranes) possessing a metallic grid basis with a great porosity. This assembly was mounted on a vacuum flask and vacuum was applied. Various filling solutions, prepared with the following polymers:
Poly(acrylic acid) sodium salt, 35% in H₂0 Mw 60.000 (PolySciences Inc);
Poly(acrylic acid) sodium salt, 35% in H₂0 Mw 250.000 (Sigma Aldrich);
Poly(acrylic acid) powder Mw 1.000.000 (PolySciences Inc);
Poly(styrenesulfonic acid-Maleic anhydride), 3:1, Low Mn (Polysciences Inc);
Poly(vinylphosphonic acid), 30% in H₂0;
PolyStyrene solid, Mw 192.000 (Sigma Aldrich); and
Poly(ethyleneglycol) Mw 950-1.050 (Sigma Aldrich),
   were loaded onto a cellulose sheet, then allowed to flow through the sheet under vacuum suction. The polymer solutions were thus penetrated into the cellulose sheet until saturation, i.e. until no outflow (or an extremely low outflow) was observed. The thus-obtained pretreated cellulose sheets were then dried at room temperature for 24 to 48 hours.

Solvents for preparing the corresponding polymer solutions were selected in accordance with the chemical nature of the tested polymers, such as to ensure the solubility thereof. Suitable solvents were aqueous, e.g. water was used for solubilizing Poly(acrylic acid) sodium salts, or organic, e.g. acetone was used as solvent for solubilizing polystyrene. In a first embodiment, cellulose sheets were impregnated with a solution containing a single polymer, or containing a mixture of at least two polymers. In another embodiment, cellulose sheets were impregnated by successive filtering with various polymer solutions. Impregnations conducted with aqueous polymer solutions could be interspersed with impregnations conducted with organic polymer solutions provided that:
i) organic solvents used for solubilizing polymers were water-miscible (such as, for instance, Tetrahydrofuran (THF), Dimethylformamide (DMF), acetone, ethanol, etc.); or
ii) cellulose sheets filtered with aqueous polymer solutions were fully dried before impregnation with water-immiscible organic polymer solutions (e.g. polystyrene solutions).

### Composition of Inks

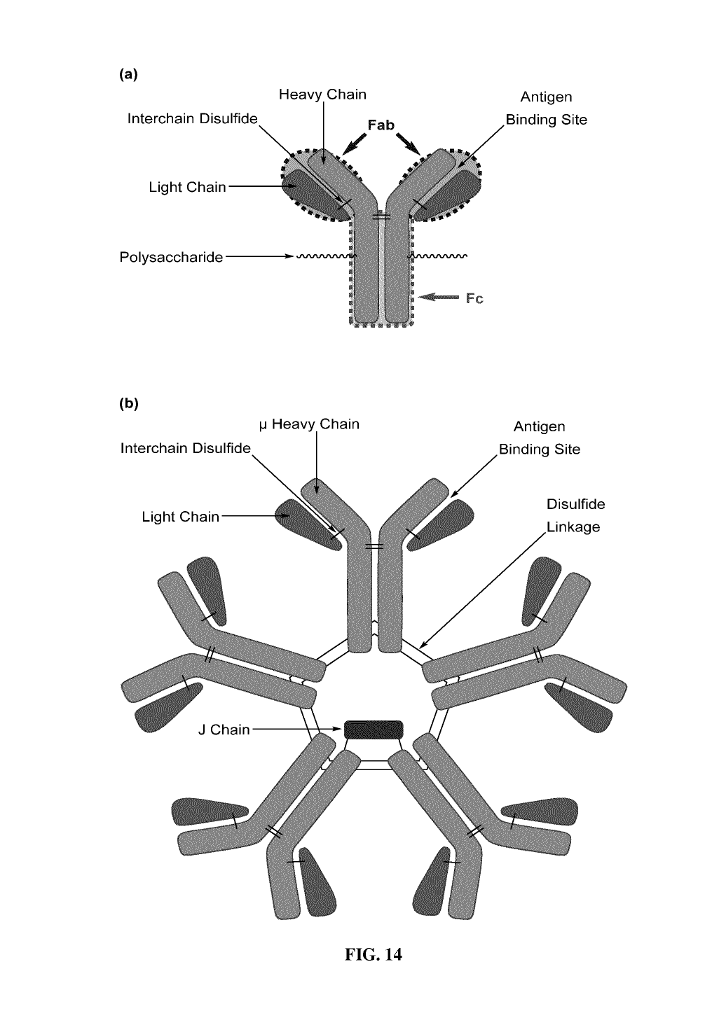

Printed solutions, also called inks, were antibody aqueous solutions. Because of different initial proportions in each antibody stock solution, their final salts content was different. Murine anti-OVA antibody solution (test line ink) contained 1 mg mL⁻¹ of monoclonal antibody (IgG) and 0.1 M of potassium phosphate in water. Goat anti-mouse antibody solution (control line ink) contained 0.5 mg mL⁻¹ of polyclonal antibody (IgG+IgM), 0.1 M of potassium phosphate and 0.05 M of sodium chloride (NaCl). Variations in salts content, as well as in antibody type (IgG and IgM structures are depicted in FIG. 14) could greatly influence the surface tension between the antibody ink and the printed paper or substrate, thereby inducing variations in the printing behavior.

### Immunochromatographic Strips

Immunochromatographic strips were prepared using Standard 14 sample wick from Whatman (Maidstone, Kent, UK), No. 470 absorbent pad from Schleicher and Schuell BioScience GmBH (Dassel, Germany) and plastic strips MIBA-020 backing card from Diagnostic Consulting Network (Carlsbad, Calif., USA). Strips were cut using an automatic programmable cutter Guillotine Cutting CM4000 Batch cutting system from BioDot (Irvine, Calif., USA). 96-Well polystyrene microplates (flat-bottom, crystal-clear, from Greiner Bio-One S.A.S. Division Bioscience, Les Ulis, France) were used as container for migrations on immunochromatographic strips.

### Antibodies Printing

Antibody solutions were either printed onto substrates using a laboratory piezoelectric drop-on-demand inkjet printer Dimatix Materials Printer DMP-2831 (Fujifilm, Santa Clara, Calif., USA) with 10 pL nominal drop volume cartridge, or dispensed at 1 μL cm⁻¹ using an automatic dispenser (XYZ3050 configured with 2 BioJet Quanti Dispenser (BioDot, Irvine, Calif., USA)).

### Photo Patterning

Opaque plastic (double-sided tape) maskings used in the photo-patterning experiments were designed and prepared with a laser plotter LaserPro Spirit (GCC Laser Pro, New Taipei City, Taiwan), and the software CorelDRAW Graphics Suite (Corel Corporation, Ottawa, Canada).

### Irradiation

Irradiations were conducted a room temperature in a U.V. chamber CN-15.LV UV viewing cabinet (Vilber Lourmat, Marne-la-Vallée, France). 96 wells plates (from Greiner Bio-One S.A.S. Division Bioscience, Les Ulis, France) were used as container for migrations on immunochromatographic strips. Colorimetric intensity resulting from colloidal gold was quantified with a Molecular imager VersaDoc MP4000, in association with the software Quantity One 1-D Analysis (Bio-Rad, Hercules, Calif., USA).

### Infrared Characterization

Infrared (IR) spectra of the various substrates were recorded on a Vertex 70 FT-IR spectrometer (Bruker, Billerica, Mass., USA) controlled by OPUS software (Bruker, Billerica, Mass., USA) and fitted with MIRacle™ ATR (Attenuated Total Reflectance) sampling accessory (PIKE Technologies, Madison, Wis., USA). The ATR crystal type was single reflection diamond/ZnSe crystal plate. The FT-IR detector was MCT working at liquid nitrogen temperature. Acquisitions were obtained at 2 cm⁻¹ resolution after 256 scans.

### X-Ray Photoelectron Spectroscopy (XPS)

XPS studies of membranes were performed with an Axis Ultra DLD spectrometer (Kratos, Manchester, UK), using monochromatic Al K_{α} radiation (1486.6 eV) at 150 W and a 90° electron take-off angle. The area illuminated by the irradiation was about 2 mm in diameter. Survey scans were recorded with 1 eV step and 160 eV analyzer pass energy and the high-resolution regions with 0.05 eV step and 40 eV analyzer pass energy. During the data acquisition, the sample surfaces were neutralized with slow thermal electrons emitted from a hot W filament and trapped above the sample by the magnetic field of the lens system (hybrid configuration). Referring to Johansson and Campbell's work, XPS analysis was carried out on dry samples, together with an in situ reference (Johansson and Campbell, Surf. Interface Anal. 2004, 36, 1018-1022).

### Microstructure and Surface Morphology

Microstructure and surface morphology of samples were examined by a JSM-5510LV (JEOL, Tokyo, Japan) scanning electron microscope (SEM) after gold coating (K575X Turbo Sputter Coater (Quorum Technologies Ltd, Ashford, Kent, UK), working at 15 mA for 20 seconds). The images were acquired at various magnifications ranging from 100× to 3 000×. The acceleration voltage and working distance were 4 kV and 17 mm, respectively. Images were acquired applying the secondary electron detector.

Surface roughness, Ra, of the unprinted substrates was measured with an AlphaStep® D-120 Stylus Profiler (KLA-Tencor, Milpitas, Calif., USA). Measurements were performed along a line of 1 mm long, with a stylus force of 1 mg and at a speed of 0.05 mm s⁻¹.

### Solution Viscosity

Printed solutions viscosity was measured before printing with a MCR 102 Rheometer (Anton Paar, Ashland, Va., USA). Cone-plane geometry was used at a shear rate varying from 100 to 10 000 s⁻¹ and at a 24° C. temperature. Gap distance was equal to 0.1 mm. Geometry diameter and angle were equal to 5 cm and 1°, respectively.

### Colloidal Gold Colorimetric Intensity

Colorimetric intensity resulting from colloidal gold on immunochromatographic strips was qualitatively estimated directly by eye at first and then indirectly through a picture taken with a Molecular Imager VersaDoc™ MP4000, in association with Quantity One 1-D Analysis software (Bio-Rad, Hercules, Calif., USA). Colorimetric intensity resulting from colloidal gold on masked papers was quantified with the same imager and software.

### Methods

### Preparation of Colloidal-Gold Labeled Antibodies

Tracer antibodies were labeled with colloidal gold according to a known method previously described (Khreich et al. Anal. Biochem. 2008, 377, 182-188). Two types of tracer were prepared: a goat anti-mouse tracer to reveal the immobilized murine antibodies, and a murine anti-OVA tracer to highlight the capture of OVA by the immobilized antibodies.

Briefly, 4 mL of gold chloride and 1 mL of 1% (w/v) sodium citrate solution were added to 40 mL of boiling water under constant stirring. Once the mixture had turned purple, this colloidal gold solution was allowed to cool down to room temperature and stored at 4° C. in the dark. 25 μg of mAb and 100 μL of 20 mM borax buffer, pH 9.3, were added to 1 mL of this colloidal gold solution. This mixture was left to incubate for one hour on a rotary shaker at room temperature, therefore enabling the ionic adsorption of the antibodies onto the surface of the colloidal gold particles. Afterwards, 100 μL of 20 mM borax buffer, pH 9.3, containing 1% (w/v) BSA, was added and the mixture was centrifuged at 15 000 g for 50 minutes at 4° C. After discarding the supernatant, the pellet was suspended in 250 μL of 2 mM borax buffer, pH 9.3, containing 1% (w/v) BSA and stored at 4° C. in the dark.

### Immobilization on Nitrocellulose

Antibodies were adsorbed onto nitrocellulose substrate (AE 98 Fast nitrocellulose). Adsorption onto nitrocellulose was generally achieved by regular 1-hour incubation at room temperature and following washing step. Nitrocellulose was used as the reference material for analyzing the immobilization of antibodies onto raw and pretreated cellulose substrates or PET substrates.

### Immobilization on Cellulose, Pretreated Cellulose and PET Substrates

Antibodies were photoimmobilized onto cellulose substrates (e.g. CF1 or Xerox premier cellulose), pretreated cellulose substrates (e.g. glucose-cellulose or paraffin-cellulose) or onto PET substrates.

The photoimmobilization process can be described as follows: (i) an antibody solution was dispensed onto a substrate sheet formed of cellulose, of pretreated cellulose or of any naturally substantially non-porous material (e.g. PET); (ii) antibodies were concentrated by drying of the impregnated substrate at 37° C., in a ventilated oven, for 15 minutes (unless otherwise specified); (iii) the system was irradiated at 365 nm (1050 μW cm⁻²) for 2 h40 (about 10 J cm⁻²) for inducing photoimmobilization (unless otherwise specified); and (iv) substrates were intensively rinsed with a washing buffer (0.1M potassium phosphate buffer, pH 7.4, containing 0.5 M NaCl and 0.5% (v/v) Tween 20) for removing non-immobilized antibodies (unless otherwise specified).

### Patterned Photoimmobilization of Probe Antibodies

Probe antibodies, or colloidal-gold-labeled antibodies (tracers), were photoimmobilized onto pristine CF1 cellulose paper according to the following procedure. A 2-cm² cellulose sheet (2 cm×1 cm in size) was manually impregnated with a goat anti-mouse tracer solution (3-fold dilution in the analysis buffer, 20 μL cm⁻² deposit). Drying step was skipped and this system was then irradiated at 365 nm for 1 h20 (about 5 J cm⁻²) through an opaque plastic mask in order to localize the grafting (patterning process). Paper was rinsed overnight with the washing buffer. Colorimetric measurement using the molecular imager was performed immediately after the paper had been slightly dried over absorbent paper. The patterned image was pictured with either digital camera or VersaDoc™ Molecular Imager.

### Inkjet Printing

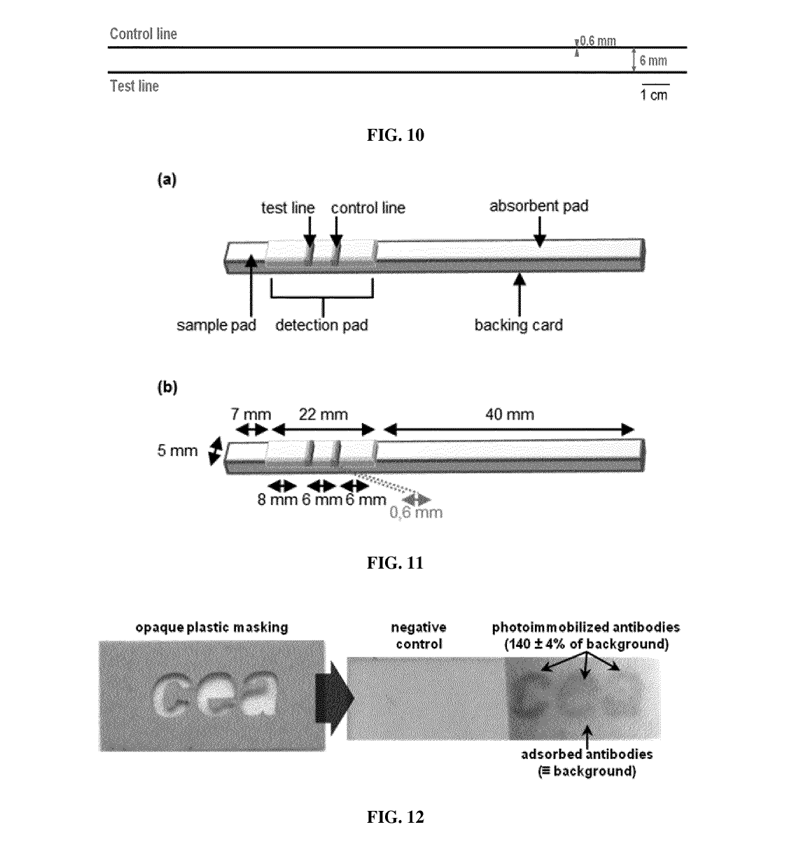

Antibody solutions were printed onto the raw or pretreated substrates using the Dimatix inkjet printer. Nozzle diameter was 21.5 μm and nominal drop volume was 10 pL. Printing tests were performed at 40 V tension with 15 μm drop spacing. While drop spacing is inversely proportionate to resolution, printing voltage is directly related to the ejected volume. The printed pattern (see FIG. 10) consisted of two straight lines of 600 μm width and was designed according to usual LFIA strips (Khreich et al., Anal. Biochem. 2008, 377, 182-188). The bottom line was dedicated to capture the OVA model antigen (test line). The top line aimed to detect anti-OVA tracer antibodies (control line). Thus, the test line consisted of murine anti-OVA monoclonal antibodies (1 mg mL⁻¹ in 0.1 M potassium phosphate buffer, pH 7.4) and the control line of goat anti-mouse polyclonal antibodies (0.5 mg mL⁻¹ in 0.1 M potassium phosphate buffer, pH 7.4). Printings made of 1 and 5 layers were compared to the usual automatic dispensing method (1 μL cm⁻¹ with the BioDot system) (Khreich et al., Anal. Biochem. 2008, 377, 182-188).

### Immunochromatographic Assays (LFIA)

Immobilization rate, biological activity and visual detection limit (VDL) of the antibody-printed membranes were evaluated by colloidal-gold-based lateral flow immunoassays (LFIAs) (Ngom et al., J. Mater. Chem. B 2013, 1, 3277-3286). The signal intensity was qualitatively estimated directly by eye at first and then indirectly through a picture taken with a Molecular Imager. All results were compared with those obtained with nitrocellulose which was regarded as the reference material.

All the reagents were diluted in the analysis buffer (0.1 M potassium phosphate buffer, pH 7.4, containing 0.1% (w/v) BSA, 0.15 M NaCl, and 0.5% (v/v) Tween 20), at room temperature, 30 minutes prior to migration in order to reduce nonspecific binding. Each assay was performed at room temperature by inserting a strip into a well of a 96-well microtiter plate containing 100 μL of the test solution. The mixture was successively absorbed by the various pads and the capillary migration process lasted for about 15 minutes. Colorimetric intensity was immediately estimated by eye and pictures with both regular digital camera and Molecular Imager were taken without delay.

### Immunochromatographic Assays for Examples 1-13

For each membrane, an antibody solution OVA1 (murine antibody directed against OVA epitopes), at a concentration of 1 mg/mL in potassium phosphate buffer 0.1M, pH 7.4, was poured on a 0.25 cm²-cellulose sheet (0.5 cm×0.5 cm), at a rate of 40 μg/cm². Where appropriate, drying was performed at 37° C., in a ventilated oven, for 15 minutes. Irradiation was generally conducted at 365 nm (1050 μW/cm² at 365 nm). After irradiation, samples were rinsed with a phosphate buffer (potassium phosphate at 0.1M, pH 7.4) optionally enriched with salts and/or detergent (for instance potassium phosphate at 0.1M, pH 7.4 containing 0.5M NaCl and 0.5% (v/v) Tween 20). Salts allow the electrostatic interactions between biomolecules and surface to be limited, and the detergent reduces or prevents hydrophobic interactions. Salts and/or detergent thus contribute to restrain biomolecules adsorption.

Membranes were saturated with a gelatin solution (potassium phosphate at 0.1M, pH 7.4, NaCl 0.15M and gelatin 0.5% (w/v) for preventing non-specific binding on membranes. Saturation was performed overnight: membranes were impregnated with the gelatin solution at 4° C., then dried at 37° C. in a ventilated oven for 30 minutes.

### Immunochromatographic Assays Performed on Immunochromatographic Strips

An immunochromatographic strip is usually composed of a sample pad, a detection pad and an absorbent pad, the whole being affixed onto a plastic carrier (or backing card). In some embodiments, the detection area is formed by antibody-grafted membranes. Migration is supported by two migration areas, surrounding the detection area, and composed of the same type of paper than the detection area, free of antibodies and saturated with gelatin. In order to prevent nonspecific protein adsorption onto the detection membrane during immunoassays, all antibody-printed membranes were indeed saturated with a gelatin solution (0.1 M potassium phosphate buffer, pH 7.4, containing 0.5% (w/v) porcine gelatin and 0.15 M NaCl) overnight at 4° C., and then dried at 37° C. in a ventilated oven for 30 minutes. All pads (about 20 cm width) were assembled onto the backing card and then the whole was cut into strips of 5 mm width (see FIG. 11).

The immunoanalysis buffer, in the context of the present experiments, was composed of a potassium phosphate buffer at 0.1M, pH 7.4, containing BSA (0.1% (w/v)), salt (NaCl 0.15M) and detergent (Tween 20 0.5% (v/v)). The concentration of the OVA starting solution was of 10 μg/mL. Tracer antibodies were labelled with colloidal gold according to known methods (Kreich et al., Analytical Biochemistry 377 (2008) 182-188). Antibodies were diluted 10 times within the analysis buffer. Solutions to be analyzed were then dispatched in the wells of 96-well plates (100 μL/well) and strips were introduced in the wells. Migration was allowed for 15 minutes, then strips were dried at 37° C. in a ventilated oven for 30 minutes. Finally, the colorimetric measure was performed right after rehydration of membranes with the analysis buffer.

### Assessment of the Immobilization

A test solution composed of a goat anti-mouse tracer diluted 10 times in the analysis buffer was used for assessing the immobilization of antibodies. Unprinted parts of detection paper pads assessed the unspecific signal due to unspecific adsorption of the tracer onto the saturating matrix during immunoassays. The immobilization ability of the various paper substrates was therefore assessed by the colorimetric difference between the murine-antibody-printed part of detection pad (test line) and the unprinted (or gelatin-grafted) corresponding one.

### Assessment of the Biological Activity and Determination of the Visual Detection Limit

Ten test solutions were prepared and pre-incubated for 15 minutes. The first one only contained murine anti-OVA mAb tracer diluted 10 times in the analysis buffer. This immunoassay without OVA antigen (0 ng mL⁻¹) assessed the unspecific signal due to unspecific adsorption of the tracer onto the antibody-gelatin matrix during immunoassays (negative control). The nine others were solutions of murine anti-OVA mAb tracer (10-time dilution) and OVA (dilution series ranging from 1 ng mL⁻¹ to 500 ng mL⁻¹) in the analysis buffer. The biological activity of the various paper substrates was therefore assessed by the colorimetric difference between the antibody-printed paper test-line signal in the presence of OVA and the corresponding one without OVA. Since it captured the excess murine anti-OVA tracer antibodies, the control line prevented false negative results. Its coloring guaranteed that the tracer actually passed through the test line, along with the test solution. The visual detection limit (VDL) was determined through the OVA dilutions series. It was defined as the minimum OVA concentration resulting in a test-line colored signal significantly more intense than the negative control one

The grafting rate and the activity rate were measured for each experimental condition.

The grafting rate of the cellulose paper was measured by establishing the difference between the antibody-grafted paper signal and the gelatin-grafted corresponding one displaying the unspecific adsorption of the goat-anti-mouse antibody labelled with colloidal gold (“tracer”) onto the gelatin matrix.

The activity rate of the grafted antibodies was measured by establishing the difference between the signal obtained in presence and in absence of OVA (ovalbumin), for the binding of the OVA35 tracer (i.e., a colloidal-gold labelled antibody directed against OVA). Non-specific adsorption of the OVA35 tracer onto the antibody gelatin matrix was measured in absence of OVA. Adsorption on cellulose only was used as a negative control and on nitrocellulose as a positive control. Considering that adsorption on nitrocellulose is the most frequently used method for immunochromatographic strips, it was herein considered as the reference and it was assimilated to 100% for both the grafting rate and the activity rate.

1-15. (canceled)

16. A process for immobilizing biomolecules on a non-functionalized carrier comprising the steps of:
(i) impregnating the non-functionalized carrier with a solution containing the said biomolecules; and
(ii) irradiating the impregnated carrier resulting from step (i) with a light of a wavelength of at least 340 nm;
wherein said biomolecules are not functionalized;
wherein the non-functionalized carrier is selected from cellulose, or the non-functionalized carrier is selected from polyethylene terephthalate (PET), polyethylene naphthalate (PEN), poly(methyl methacrylate) (PMMA), polyurethane (PU), poly(vinyl chloride) (PVC), polyethylene (PE), polystyrene (PS), polylactate, polyamide, and combinations thereof;
wherein the process further comprises a preliminary step, before step (i), of rendering the said non-functionalized carrier substantially non-porous, comprising the steps of:
a) impregnating said non-functionalized carrier with at least one filler until saturation of the carrier, and
b) drying the impregnated non-functionalized carrier resulting from step a).

17. The process according to claim 16, further comprising a step of drying said impregnated carrier after step (i) and before step (ii).

18. The process according to claim 16, further comprising at least one step of washing the irradiated carrier resulting from step (ii).

19. The process according to claim 16, wherein the light used for irradiating the impregnated carrier has a wavelength of from 340 nm to 800 nm.

20. The process according to claim 16, wherein said impregnated carrier is irradiated during step (ii) with a photoenergy of from 1 mJ/cm² to 500 J/cm².

21. The process according to claim 16, wherein said non-functionalized carrier is substantially non-porous.

22. The process according to claim 16, wherein said non-functionalized carrier is cellulose and wherein said at least one filler is selected in the group consisting of glucose, paraffin, sulfonated polymers, polyacrylic acid (PAA), poly-2-hydroxyethyl methacrylate (PHEMA), polymethyl methacrylate (PMMA), poly(ethylene glycol) dimethacrylate (PPEGDMA), polypropylene (PP), polys(styrene sulfonic acid-maleic anhydride), poly(vinyl phosphonic acid), polyethyleneglycol, salts thereof and/or combinations thereof.

23. The process according to claim 16, wherein the said non-functionalized carrier is in a form selected in the group consisting of a bead, a well, a sheet, a powder, a stick, a plate, a strip or a tube.

24. The process according to claim 16, wherein said biomolecule is selected from the group consisting of proteins or peptides, such as antibodies, antigens, enzymes, transcription factors, protein domains or binding proteins.

25. The process according to claim 16, wherein said biomolecule is displayed on the surface of a bacteria, a virus or a micro-organism, or is free in solution.

26. The process according to claim 18, wherein the buffer used for washing the irradiated carrier is selected from the group consisting of water, phosphate buffer, carbonate buffer, borate buffer, HEPES buffer, MES buffer or any other aqueous biological buffer, and further optionally comprises salts and/or detergent.

27. A grafted carrier obtained by the process of claim 16, wherein said carrier comprises biomolecules immobilized thereonto.

28. The grafted carrier of claim 27, being comprised in a bioassay device.

29. The grafted carrier of claim 27, being comprised in an immunoassay device such as an immunochromatographic strip or an immunochromatographic multiplex system.

30. A method for diagnosis, affinity chromatography, proteomics, genomics and/or drug screening, comprising detecting and/or quantifying biological or non-biological compounds, objects or organisms using at least one grafted carrier according to claim 27.
